(19) United States
(12) Patent Application Publication
(10) Pub. No.: US 2019/0316966 A1
(43) Pub. Date: Oct. 17, 2019

(54) IMAGE PROCESSING DEVICE AND IMAGE PROCESSING METHOD

(71) Applicant: SONY CORPORATION, TOKYO (JP)

(72) Inventors: MASAYUKI YOKOYAMA, TOKYO (JP); YUKI ASUKABE, SAITAMA (JP); ATSUSHI ITO, KANAGAWA (JP); HIDEKI OYAIZU, TOKYO (JP)

(21) Appl. No.: 16/314,616

(22) PCT Filed: Mar. 31, 2017
(86) PCT No.: PCT/JP2017/013701

§ 371 Date: Dec. 31, 2018

(30) Foreign Application Priority Data
Jul. 13, 2016 (JP) 2016-138848

(51) Int. Cl. G01J 5/10; G01J 5/00; G01V 8/10; G06K 9/00; G06T 7/00; H04N 5/33; G06T 7/70; A61B 5/01

(52) U.S. Cl. CPC G01J 5/10; G01J 5/0025; G01V 8/10; G06K 9/00228; G06K 9/00369; G06K 9/00791; G06T 2207/30252; H04N 5/33; G06T 7/70; A61B 5/015; G01J 2005/0077; G06T 2207/10048; G06T 2207/30196; G06T 7/0012

(57) ABSTRACT

[Object] To improve detection precision of a subject at a lower cost. [Solution] Provided is an image processing device including: a plurality of detection units configured to detect respective detection regions indicating temperatures within mutually different setting temperature ranges from a far-infrared image; and a determination unit configured to determine whether a predetermined subject is shown in the far-infrared image on the basis of a positional relation between the plurality of detected detection regions and modeling which regulates the positional relation in a case in which the predetermined subject is shown in the far-infrared image.

### TECHNICAL FIELD

The present disclosure relates to an image processing device and an image processing method.

### BACKGROUND ART

In recent years, far-infrared images have been used to detect temperatures of objects or the like. Far-infrared images are generated when imaging elements capture far-infrared rays emitted from objects by black-body radiation. By using such far-infrared images, subjects can be detected even in some cases in which it is difficult to detect subjects such as human bodies from visible-light images, for example, at night or in bad weather. However, far-infrared images may not be obtained with desired detection precision in detection of subjects due to lower resolutions or the like than those of visible-light images in some cases. Accordingly, technologies for improving detection precision of subjects have been proposed.

For example, Patent Literature 1 proposes a technology for preventing precision of determination of types of objects from decreasing in a case in which reliability of images captured by a camera deteriorates due to an influence of an environment. In the technology, a radar that is mounted on a vehicle and detects a relative position between the vehicle and an object located within a first monitoring range around the vehicle, an infrared camera that is mounted on the vehicle and images a second monitoring range overlapping the first monitoring range, and a type determination unit that determines types of objects located around the vehicle on the basis of data detected by the radar and images captured by the infrared camera are included. The type determination unit excludes types of objects which can be determined on the basis of the images captured by the infrared camera, and then determines types of objects which are not determined on the basis of the data detected by the radar.

### CITATION LIST

### Patent Literature

Patent Literature 1: JP 2014-209387A

### DISCLOSURE OF INVENTION

### Technical Problem

Incidentally, in fields related to detection of subjects, it is considered preferable to improve detection precision of subjects at a lower cost. For example, in the technology disclosed in Patent Literature 1, the radar is used in addition to the infrared camera. Therefore, cost may increase in detection of subjects.

Accordingly, the present disclosure proposes a novel and improved image processing device and a novel and improved image processing method capable of improving detection precision of a subject at a lower cost.

### Solution to Problem

According to the present disclosure, there is provided an image processing device including: a plurality of detection units configured to detect respective detection regions indicating temperatures within mutually different setting temperature ranges from a far-infrared image; and a determination unit configured to determine whether a predetermined subject is shown in the far-infrared image on the basis of a positional relation between the plurality of detected detection regions and modeling which regulates the positional relation in a case in which the predetermined subject is shown in the far-infrared image.

In addition, according to the present disclosure, there is provided an image processing method including: detecting respective detection regions indicating temperatures within mutually different setting temperature ranges from a far-infrared image; and determining whether a predetermined subject is shown in the far-infrared image by an image processing device on the basis of a positional relation between the plurality of detected detection regions and modeling which regulates the positional relation in a case in which the predetermined subject is shown in the far-infrared image.

### Advantageous Effects of Invention

According to the present disclosure, as described above, it is possible to improve detection precision of a subject at a lower cost.

Note that the effects described above are not necessarily limitative. With or in the place of the above effects, there may be achieved any one of the effects described in this specification or other effects that may be grasped from this specification.

### BRIEF DESCRIPTION OF DRAWINGS

FIG. 1 is an explanatory diagram illustrating various uses of an infrared image which depends on a wavelength.

FIG. 2 is a block diagram illustrating an example of a hardware configuration of an image processing device according to an embodiment of the present disclosure.

FIG. 3 is a block diagram illustrating an example of a functional configuration of the image processing device according to the embodiment.

FIG. 4 is a flowchart illustrating an example of a flow of a process performed by the image processing device according to the embodiment.

FIG. 5 is a flowchart illustrating an example of a flow of a detection process for each candidate region performed by the image processing device according to the embodiment.

FIG. 6 is a block diagram illustrating an example of a functional configuration of the image processing device according to a first application example.

FIG. 7 is an explanatory diagram illustrating an example of a far-infrared image in which human bodies are shown.

FIG. 8 is an explanatory diagram illustrating an example of a data table stored in a storage unit.

FIG. 9 is an explanatory diagram illustrating an example of a calculation result of a score value by each detection unit.

FIG. 10 is an explanatory diagram illustrating an example of an image after conversion from a pixel value of each pixel into a likelihood.

FIG. 11 is a flowchart illustrating a first example of a flow of a process performed by the image processing device according to the first application example.

FIG. 12 is a flowchart illustrating an example of a flow of a detection process for each of a face candidate region and a trunk candidate region performed by the image processing device according to the first application example.

FIG. 13 is a flowchart illustrating a second example of a flow of a process performed by the image processing device according to the first application example.

FIG. 14 is a flowchart illustrating an example of a flow of a detection process for a face part candidate region performed by the image processing device according to the first application example.

FIG. 15 is a block diagram illustrating an example of a functional configuration of the image processing device according to a second application example.

FIG. 16 is an explanatory diagram illustrating an example of a far-infrared image in which a vehicle is shown.

FIG. 17 is an explanatory diagram illustrating an example of a data table stored in a storage unit.

FIG. 18 is an explanatory diagram illustrating an example of a calculation result of a score value by each detection unit.

FIG. 19 is a flowchart illustrating an example of a flow of a process performed by the image processing device according to the second application example.

FIG. 20 is a flowchart illustrating an example of a flow of a detection process for each of a non-passage face candidate region, a passage candidate region, and a muffler candidate region performed by the image processing device according to the second application example.

FIG. 21 is a block diagram illustrating an example of a functional configuration of the image processing device according to a third application example.

FIG. 22 is an explanatory diagram illustrating a surgery in which a microscopic device is used.

FIG. 23 is an explanatory diagram illustrating an example of a far-infrared image in which an abnormal part in an opened abdominal part of a patient is shown.

FIG. 24 is an explanatory diagram illustrating an example of a data table stored in the storage unit.

FIG. 25 is an explanatory diagram illustrating an example of a calculation result of a score value by each detection unit.

FIG. 26 is a flowchart illustrating an example of a flow of a process performed by the image processing device according to the third application example.

FIG. 27 is a flowchart illustrating an example of a flow of a detection process for a body surface candidate region, an opened abdominal part candidate region, and an abnormal part candidate region performed by the image processing device according to the third application example.

### MODE(S) FOR CARRYING OUT THE INVENTION

Hereinafter, (a) preferred embodiment(s) of the present disclosure will be described in detail with reference to the appended drawings. Note that, in this specification and the appended drawings, structural elements that have substantially the same function and structure are denoted with the same reference numerals, and repeated explanation of these structural elements is omitted.

Note that the description will be made in the following order.

### 1. Introduction

2. Overview of image processing device
2-1. Hardware configuration
2-2. Functional configuration

### 2-3. Operation

3. Application examples
3-1. First application example
3-2. Second application example
3-3. Third application example

### 4. Conclusion

### 1. INTRODUCTION

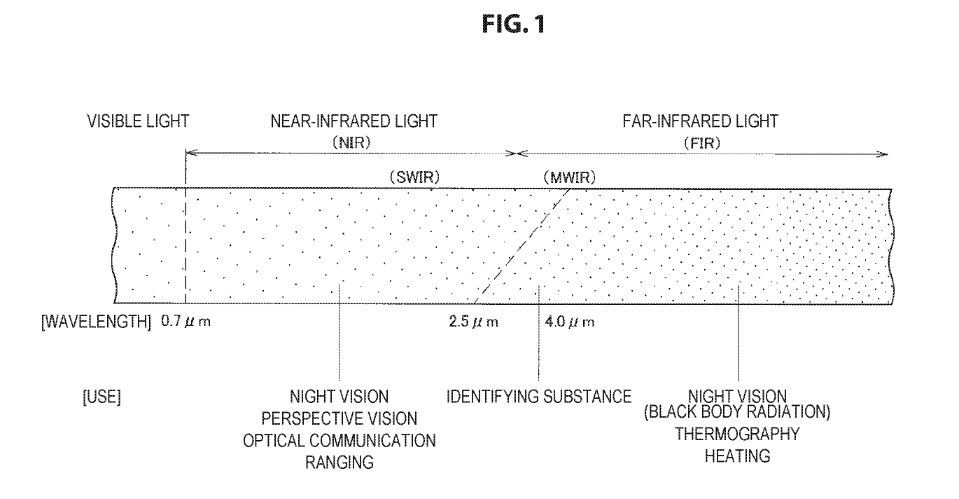

FIG. 1 is an explanatory diagram illustrating various applications of an infrared image which depends on a wavelength. The horizontal direction of FIG. 1 corresponds to a wavelength of the infrared light and the wavelength is lengthened from the left to the right. Light that has a wavelength equal to or less than 0.7 μm is visible light and a human visual sense detects the visible light. A wavelength region adjacent to the visible light region is a near-infrared light region (NIR) and the infrared light belonging to the NIR region is referred to as near-infrared light. The upper limit of the wavelength of the NIR region differs depending on definition and is between 2.5 μm to 4.0 μm in many cases. A portion with a relatively long wavelength in the NIR region is also referred to as a short-wavelength infrared (SWIR) region in some cases. The near-infrared light can be used for, for example, night vision, perspective vision, optical communication, and distance measurement. Normally, a camera that captures a near-infrared image first emits infrared light to the vicinity and captures reflected light. The wavelength region adjacent to the NIR region on a long-wavelength side is a far-infrared (FIR) region and infrared ray belonging to the FIR region is referred to as far-infrared light. A portion with a relatively short wavelength in the FIR region is also referred to as a middle-wavelength infrared (MWIR) region in some cases. Since an absorption spectrum specific to a substance is shown in a wavelength range of the middle-wavelength infrared light, middle-wavelength infrared light can be used to identify the substance. The far-infrared light can be used for night vision, thermography, and heating. The infrared light emitted by black body radiation from a substance is equivalent to the far-infrared light. Therefore, a night vision device that uses the far-infrared light can generate a far-infrared image by trapping the black body radiation from the substance even when the night vision device does not emit the infrared light. Note that the boundary values of the ranges of the wavelengths illustrated in FIG. 1 are merely exemplary. There are various definitions for the boundary values of classification of the infrared light, and thus the advantages of the technology according to the present disclosure to be described below can be gained under any definition.

Since energy of the far-infrared light radiated from a substance and a temperature of the substance have a correlation, a temperature difference between a plurality of substances shown in a far-infrared image can be detected from the far-infrared image generated by receiving the far-infrared light. Thus, a region in which a specific object is shown can be distinguished from other regions to be extracted from the far-infrared image. For example, since a temperature of a living body shown in the far-infrared image is generally higher than a temperature of objects near the living body, a living body region can be extracted from the far-infrared image by detecting a temperature difference between the living body and the nearby objects. In addition, by using image processing such as template matching for the extracted region, it is possible to detect a subject corresponding to the region. Therefore, it is possible to determine the kind of subject corresponding to the region.

However, a far-infrared image generally tends to have a lower resolution than a visible image. Therefore, when a subject is detected using the foregoing image processing, desired detection precision may not be obtained. Here, as described above, detection precision of a subject is considered to be improved by acquiring different data from a far-infrared image using another device different from an infrared camera and using the far-infrared image and the data in combination. However, according to such a method, the other device is used in addition to the infrared camera. Therefore, the cost may increase. Accordingly, the present specification proposes a structure capable of improving detection precision of a subject at a lower cost.

### 2. OVERVIEW OF IMAGE PROCESSING DEVICE

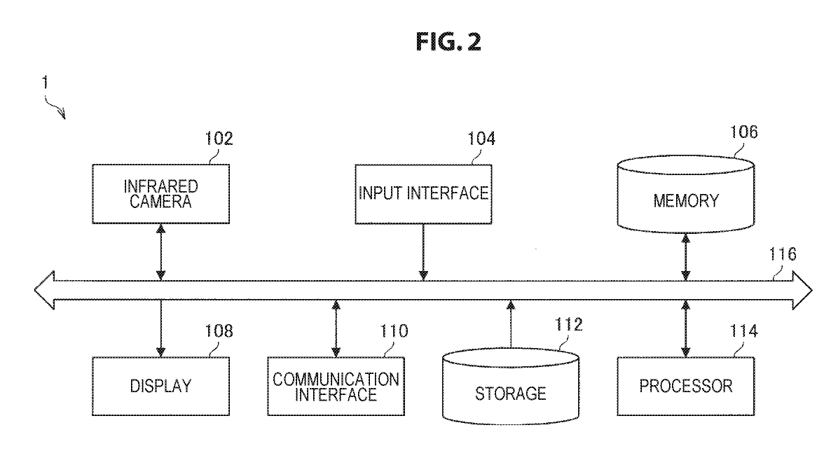

Next, an overview of an image processing device 1 according to an embodiment of the present disclosure will be described with reference to FIGS. 2 to 5.

### [2-1. Hardware Configuration]

First, a hardware configuration of the image processing device 1 according to the embodiment will be described with reference to FIG. 2.

FIG. 2 is a block diagram illustrating an example of a hardware configuration of the image processing device 1 according to the embodiment. As illustrated in FIG. 2, the image processing device 1 includes an infrared camera 102, an input interface 104, a memory 106, a display 108, a communication interface 110, a storage 112, a processor 114, and a bus 116.

### (Infrared Camera)

The infrared camera 102 is an imaging module that performs imaging using infrared light and obtains an infrared image which is a non-color image. The infrared camera 102 is equivalent to an imaging unit according to the present disclosure. Specifically, the infrared camera 102 has an array of an imaging element that detects far-infrared light with wavelengths belonging to the FIR region and captures a far-infrared image. For example, the infrared camera 102 captures far-infrared images at a given time interval. In addition, a series of far-infrared images obtained from the infrared camera 102 may form a video.

### (Input Interface)

The input interface 104 is used by the user to manipulate the image processing device 1 or input information to the image processing device 1. For example, the input interface 104 may include an input device such as a touch sensor, a keyboard, a keypad, a button, or a switch. In addition, the input interface 104 may include a voice input microphone and a voice recognition module. In addition, the input interface 104 may include a remote manipulation module that receives a command selected by the user from a remote device.

### (Memory)

The memory 106 is a storage medium that can include a random access memory (RAM) and a read-only memory (ROM). The memory 106 is connected to the processor 114 and stores data and a program used for a process performed by the processor 114.

### (Display)

The display 108 is a display module that has a screen on which an image is displayed. For example, the display 108 may be a liquid crystal display (LCD), an organic light-emitting diode (OLED), a cathode ray tube (CRT), or the like.

### (Communication Interface)

The communication interface 110 is a module that relays communication between the image processing device 1 and another device. The communication interface 110 establishes communication connection in conformity with any wireless communication protocol or wired communication protocol.

### (Storage)

The storage 112 is a storage device that accumulates infrared image data or stores a database used for image processing. The storage 112 contains a storage medium such as a semiconductor memory or a hard disk. Note that data and a program to be described in the present specification may be acquired from an external data source (for example, a data server, a network storage, an externally attached memory, or the like) of the image processing device 1.

### (Processor)

The processor 114 is a processing module such as a central processing unit (CPU) or a digital signal processor (DSP). The processor 114 causes a function to be operated in order to improve detection precision of a subject at a lower cost by executing a program stored in the memory 106 or another storage medium.

### (Bus)

The bus 116 connects the infrared camera 102, the input interface 104, the memory 106, the display 108, the communication interface 110, the storage 112, and the processor 114 to each other.

### [2-2. Functional Configuration]

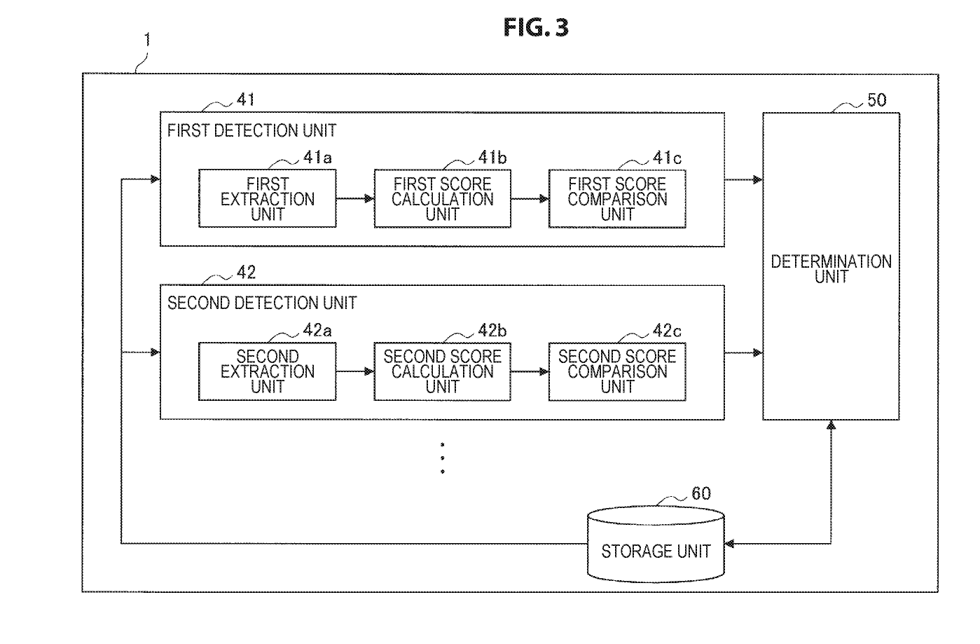

Next, a functional configuration of the image processing device 1 according to the embodiment will be described with reference to FIG. 3. Note that, hereinafter, an overview of a process performed by each functional configuration will be described and details of a process performed by each functional configuration will be described in each application example to be described below.

FIG. 3 is a block diagram illustrating an example of a functional configuration realized by mutual cooperation between constituent elements of the image processing device 1 illustrated in FIG. 2. As illustrated in FIG. 3, the image processing device 1 includes a first detection unit 41, a second detection unit 42, a determination unit 50, and a storage unit 60. The first detection unit 41 and the second detection unit 42 illustrated in FIG. 3 are equivalent to a plurality of detection units according to the present disclosure. Note that the image processing device 1 according to the embodiment may include at least two detection units and the number of detection units illustrated in FIG. 3 is merely exemplary.

### (Storage Unit)

The storage unit 60 stores data which is referred to in each process performed by the image processing device 1. For example, the storage unit 60 stores information which is used in a detection process for the candidate region performed by each of the first detection unit 41 and the second detection unit 42. In addition, the storage unit 60 stores modeling which is used in the determination process performed by the determination unit 50. The modeling is an index which is used to determine whether or not a predetermined subject is shown in a far-infrared image. In addition, the storage unit 60 may store a far-infrared image for each frame captured by the infrared camera 102. Each functional unit can acquire a far-infrared image captured by the infrared camera 102 from the storage unit 60. In addition, each functional unit may directly acquire a far-infrared image from the infrared camera 102. In addition, each functional unit may acquire a far-infrared image from another device via the communication interface 110.

### (First Detection Unit)

The first detection unit 41 detects a first detection region indicating a temperature within a first setting temperature range from the far-infrared image. In addition, the first detection unit 41 outputs a detection result to the determination unit 50. The first setting temperature range is a temperature range in accordance with an assumed temperature of a first target corresponding to the first detection unit 41. The assumed temperature is a temperature assumed as a general temperature of the first target. In addition, the first detection region indicating the temperature within the first setting temperature range is equivalent to a first candidate region which is a candidate in which the first target is shown. Therefore, the first setting temperature range is equivalent to a range of a temperature indicated by a region in which there is a relatively high possibility of the first target being shown in the far-infrared image. In other words, the first detection unit 41 detects the first candidate region from the far-infrared image in this way. The first detection unit 41 includes, for example, a first extraction unit 41a, a first score calculation unit 41b, and a first score comparison unit 41c, as illustrated in FIG. 3.

The first extraction unit 41a extracts a partial region from the far-infrared image and outputs an extraction result to the first score calculation unit 41b. The partial region is, for example, rectangular and has predetermined dimensions. The predetermined dimensions are set in accordance with the first target. Specifically, the predetermined dimensions are substantially identical to dimensions assumed as dimensions of a region in which the first target in the far-infrared image is shown. Note that information indicating the predetermined dimensions can be stored in the storage unit 60. As will be described below, in a case in which the partial region satisfies a predetermined condition, the first detection unit 41 detects the partial region as a first candidate region. Therefore, the first detection unit 41 can detect a region that has the predetermined dimensions set in accordance with the first target as the first candidate region. Thus, it is possible to further increase a possibility of the first target being shown in the detected first candidate region. In addition, for example, the first extraction unit 41a repeatedly extracts a partial region so that the entire region of the far-infrared image is scanned. Then, information indicating each of the extracted partial regions is output as an extraction result to the first score calculation unit 41b.

The first score calculation unit 41b calculates a score value as a likelihood that a temperature indicated by the extracted partial region is an assumed temperature of the first target and outputs a calculation result to the first score comparison unit 41c. Specifically, the first score calculation unit 41b may calculate the score value on the basis of a probability density function corresponding to the assumed temperature of the first target. The probability density function regulates a relation between the likelihood and the temperature indicated by the partial region. In addition, as the probability density function, for example, a probability density function in accordance with a Gauss distribution of which a middle value is identical to the assumed temperature can be used. Note that in a case in which the assumed temperature has a width, for example, a probability density function in accordance with the Gauss distribution of which the middle value is identical to a middle value of the assumed temperature can be used as the probability density function. Specifically, the first score calculation unit 41b can calculate an average value of temperatures corresponding to pixel values of pixels within a partial region as a temperature indicated by the partial region and calculate a likelihood corresponding to the average value in the probability density function as a score value.

Note that information indicating the probability density function or the assumed temperature can be stored in advance in the storage unit 60. In addition, the first score calculation unit 41b may generate a probability density function on the basis of the assumed temperature. In addition, in a case in which the assumed temperature has a width and has only one of a lower limit and an upper limit, a probability density function that regulates a relation in which the likelihood is larger as the temperature indicated by the partial region is larger or smaller can be used as the probability density function.

The first score comparison unit 41c compares the calculated score value to a threshold. The threshold can be appropriately set in accordance with various design specifications or the like of the image processing device 1 such as a variation in light reception sensitivity between a plurality of imaging elements in the infrared camera 102. In a case in which the score value is greater than the threshold, the first detection unit 41 detects a corresponding partial region as the first candidate region. Conversely, in a case in which the score value is equal to or less than the threshold, the first detection unit 41 does not detect the corresponding partial region as the first candidate region. Thus, a more likely region can be detected as the first candidate region which is a candidate in which the first target is shown.

The first detection unit 41 detects the first candidate region in the entire region of the far-infrared image by repeating the process of extracting the partial region in the first extraction unit 41a, the process of calculating the score value in the first score calculation unit 41b, and the process of comparing the score value to the threshold in the first score comparison unit 41c.

### (Second Detection Unit)

The second detection unit 42 detects the second detection region indicating a temperature within a second setting temperature range different from the first setting temperature range from the far-infrared image. In addition, the second detection unit 42 outputs a detection result to the determination unit 50. The second setting temperature range is a temperature range in accordance with an assumed temperature of a second target corresponding to the second detection unit 42. Note that the second target is a different target from the first target. The assumed temperature is a temperature assumed as a general temperature of the second target. In addition, the second detection region indicating the temperature within the second setting temperature range is equivalent to a second candidate region which is a candidate in which the second target is shown. Therefore, the second setting temperature range is a temperature range in which it is possible to determine whether or not a possibility of the second target being shown in a region in the far-infrared image is relatively high. In other words, the second detection unit 42 detects the second candidate region from the far-infrared image in this way. The second detection unit 42 includes, for example, a second extraction unit 42a, a second score calculation unit 42b, and a second score comparison unit 42c, as illustrated in FIG. 3.

The second extraction unit 42a, the second score calculation unit 42b, and the second score comparison unit 42c of the second detection unit 42 correspond to the first extraction unit 41a, the first score calculation unit 41b, and the first score comparison unit 41c of the first detection unit 41 and can perform the same processes. Specifically, the second extraction unit 42a extracts a partial region from the far-infrared image and outputs an extraction result to the second score calculation unit 42b. The second score calculation unit 42b calculates a score value as a likelihood that a temperature indicated by the extracted partial region is an assumed temperature of the second target and outputs a calculation result to the second score comparison unit 42c. The second score comparison unit 42c compares the calculated score value to a threshold. In a case in which the score value is greater than the threshold, the second detection unit 42 detects a corresponding partial region as the second candidate region. Conversely, in a case in which the score value is equal to or less than the threshold, the second detection unit 42 does not detect the corresponding partial region as the second candidate region.

Note that in the partial region extraction process performed by the second extraction unit 42a, dimensions of the partial region can be set in accordance with the second target. In addition, a probability density function used in the score value calculation process performed by the second score calculation unit 42b can correspond to the assumed temperature of the second target. In addition, the threshold used in the comparison process performed by the second score comparison unit 42c may be identical to or may be different from the threshold used by the first score comparison unit 41c.

### (Determination Unit)

The determination unit 50 determines whether or not a predetermined subject is shown in a far-infrared image on the basis of modeling and a positional relation between the plurality of detected detection regions. The modeling is an index used to determine whether or not the predetermined subject is shown in the far-infrared image, as described above. Specifically, the modeling regulates a positional relation between the plurality of detection regions in a case in which the predetermined subject is shown in the far-infrared image.

Specifically, the determination unit 50 determines whether or not the predetermined subject is shown in the far-infrared image on the basis of the modeling and a positional relation between the first candidate region detected by the first detection unit 41 and the second candidate region detected by the second detection unit 42. Here, there are the first target and the second target to correspond to the predetermined subject and have a predetermined positional relation. For example, the first target and the second target may be parts in the predetermined subject. In addition, the first target and the second target may be different objects from the predetermined subject. The modeling regulates the positional relation between these objects assumed with regard to the predetermined subject as a positional relation between candidate regions in a case in which the predetermined subject is shown in the far-infrared image.

More specifically, the determination unit 50 determines that the positional relation between the candidate regions is adequate in a case in which the positional relation between the candidate regions is substantially identical to the positional relation regulated by the modeling. Then, the determination unit 50 determines that the predetermined subject is shown in the far-infrared image since the determination unit 50 determines that the positional relation between the candidate regions is adequate.

In this way, in the embodiment, whether or not the predetermined subject is shown in the far-infrared image is determined on the basis of the modeling and the positional relation between the plurality of detection regions indicating temperatures within mutually different setting temperature ranges. Therefore, when it is determined whether or not the predetermined subject is shown in the far-infrared image, a more likely determination result can be obtained. Consequently, detection precision of the subject can be improved without using another device different from the infrared camera 102. Accordingly, it is possible to improve the detection precision of the subject at a lower cost.

In addition, the determination unit 50 may output a determination result. For example, the determination unit 50 may register the determination result by outputting the determination result to the storage unit 60. In addition, the determination unit 50 may report the determination result by outputting the determination result to the display 108. In addition, the determination unit 50 may output the determination result to an external device via the communication interface 110.

In addition, the determination unit 50 may decide whether or not a determination process of determining whether or not the predetermined subject is shown in the far-infrared image is executed in accordance with detection results output from the first detection unit 41 and the second detection unit 42. For example, the determination unit 50 may perform the determination process in a case in which both the first candidate region and the second candidate region are detected. Conversely, the determination unit 50 may not perform the determination process in a case in which at least one of the first candidate region or the second candidate region is not detected.

### [2-3. Operation]

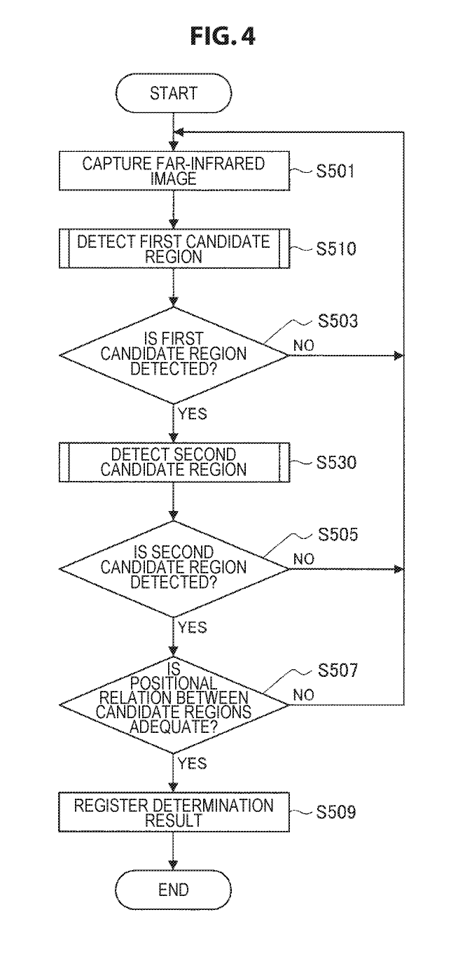

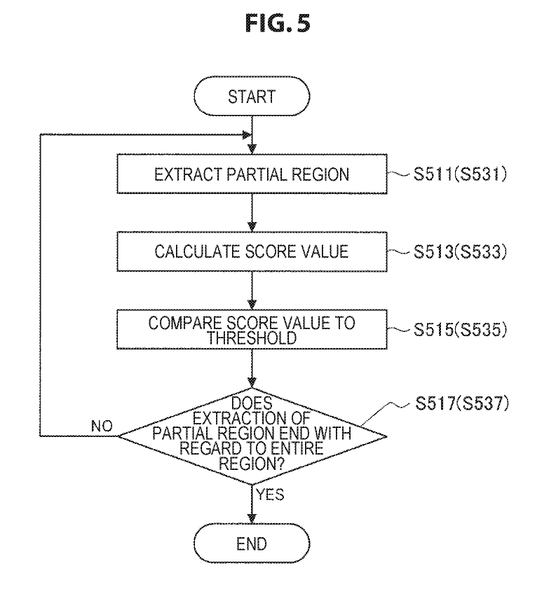

Next, a flow of a process performed by the image processing device 1 according to the embodiment will be described with reference to FIGS. 4 and 5. FIG. 4 is a flowchart illustrating an example of the flow of the process performed by the image processing device 1 according to the embodiment. The process illustrated in FIG. 4 can be performed on, for example, each frame.

As illustrated in FIG. 4, the image processing device 1 first captures a far-infrared image (step S501). Subsequently, the first detection unit 41 performs a detection process of detecting the first candidate region from the captured far-infrared image (step S510) and outputs a detection result to the determination unit 50. Then, the determination unit 50 determines whether or not the first candidate region is detected (step S503). In a case in which it is determined that the first candidate region is not detected (NO in step S503), the process returns to step S501.

Conversely, in a case in which it is determined that the first candidate region is detected (YES in step S503), a determination result is output from the determination unit 50 to the second detection unit 42. The second detection unit 42 performs a detection process of detecting the second candidate region (step S530) and outputs a detection result to the determination unit 50. Then, the determination unit 50 determines whether or not the second candidate region is detected (step S505). In a case in which it is determined that the second candidate region is not detected (NO in step S505), the process returns to step S501.

Conversely, in a case in which it is determined that the second candidate region is detected (YES in step S505), the determination unit 50 determines whether or not the positional relation between the candidate regions is adequate (step S507). In a case in which it is determined that the positional relation between the candidate regions is not substantially identical to the positional relation regulated by the modeling, it is determined that the positional relation between the candidate regions is not adequate (NO in step S507) and the process returns to step S501. Conversely, in a case in which it is determined that the positional relation between the candidate regions is substantially identical to the positional relation regulated by the modeling, it is determined that the positional relation between the candidate regions is adequate (YES in step S507). The determination unit 50 registers a determination result in the storage unit 60 (step S509) and ends the process illustrated in FIG. 4.

Next, the detection processes (steps S510 and S530 illustrated in FIG. 4) for the candidate region performed by the image processing device 1 according to the embodiment will be described in more detail with reference to FIG. 5. FIG. 5 is a flowchart illustrating an example of a flow of a detection process for each candidate region performed by the image processing device 1 according to the embodiment.

As illustrated in FIG. 5, the first extraction unit 41a (the second extraction unit 42a) first extracts a partial region from the far-infrared image (step S511 (step S531)) and outputs an extraction result to the first score calculation unit 41b (the second score calculation unit 42b). Subsequently, the first score calculation unit 41b (the second score calculation unit 42b) calculates a score value of the extracted partial region (step S513 (step S533)) and outputs a calculation result to the first score comparison unit 41c (the second score comparison unit 42c). Subsequently, the first score comparison unit 41c (the second score comparison unit 42c) compares the calculated score value to the threshold (step S515 (step S535)). Then, the first detection unit 41 (the second detection unit 42) determines whether or not the extraction of the partial region ends with regard to the entire region of the far-infrared image (step S517 (step S537)). In a case in which it is determined that the extraction of the partial region does not end with regard to the entire region of the far-infrared image (NO in step S517 (NO in step S537)), the process returns to step S511 (step S531). Conversely, in a case in which it is determined that the extraction of the partial region ends with regard to the entire region of the far-infrared image (YES in step S517 (YES in step S537)), the process illustrated in FIG. 5 ends.

### 3. APPLICATION EXAMPLES

Next, various application examples in which a technology according to the present disclosure described above is applied to detection of various subjects will be described.

### 3-1. First Application Example

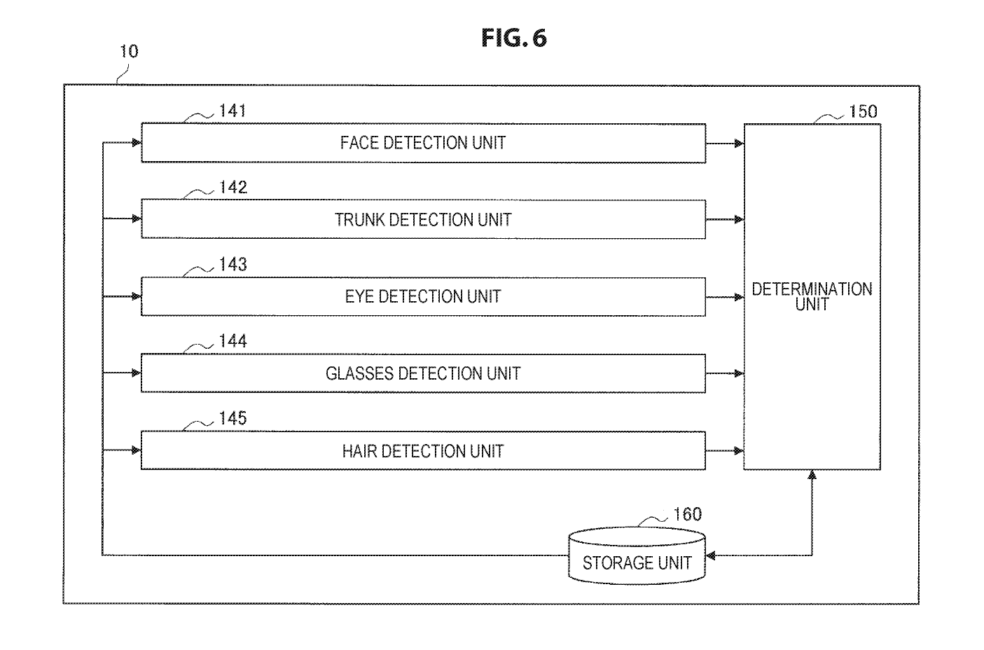

First, an image processing device 10 according to a first application example will be described with reference to FIGS. 6 to 14. The first application example is an example in which the technology according to the present disclosure is applied to detection of a human body which is a subject. The image processing device 10 according to the first application example determines whether or not a human body is shown as a predetermined subject in a far-infrared image.

### (Functional Configuration)

First, a functional configuration of the image processing device 10 according to the first application example will be described with reference to FIG. 6. A hardware configuration of the image processing device 10 according to the first application example may be similar to a hardware configuration of the image processing device 1 described with reference to FIG. 2. FIG. 6 is a block diagram illustrating an example of a functional configuration realized by mutually cooperating between constituent elements of the image processing device 10.

As illustrated in FIG. 6, the image processing device 10 includes a face detection unit 141, a trunk detection unit 142, an eye detection unit 143, a glasses detection unit 144, a hair detection unit 145, a determination unit 150, and a storage unit 160. The face detection unit 141, the trunk detection unit 142, the eye detection unit 143, the glasses detection unit 144, and the hair detection unit 145 in the first application example are equivalent to a plurality of detection units according to the present disclosure. In addition, the determination unit 150 and the storage unit 160 in the first application example respectively correspond to the determination unit 50 and the storage unit 60 of the image processing device 1 described with reference to FIG. 3.

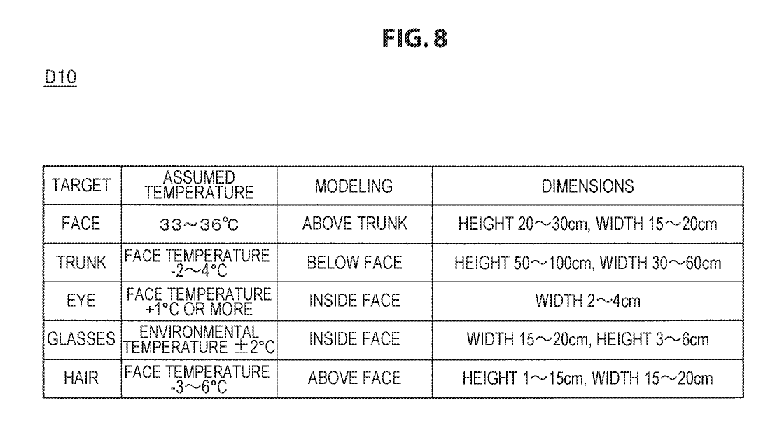

The storage unit 160 stores data which is referred to in each process performed by the image processing device 10. For example, the storage unit 160 stores information which is used in a detection process for the candidate region performed by each of the face detection unit 141, the trunk detection unit 142, the eye detection unit 143, the glasses detection unit 144, and the hair detection unit 145. In addition, the storage unit 160 stores modeling which is used in a determination process performed by the determination unit 150. Specifically, the storage unit 160 stores a data table D10 to be described below with reference to FIG. 8 and various kinds of information are included in the data table D10.

The face detection unit 141 detects a face candidate region which is a candidate in which the face of a human body is shown as a candidate region from the far-infrared image. In addition, the trunk detection unit 142 detects a trunk candidate region which is a candidate in which the trunk of the human body is shown as a candidate region from the far-infrared image. In addition, the eye detection unit 143 detects an eye candidate region which is a candidate in which an eye of the human body is shown as a candidate region from the far-infrared image. In addition, the glasses detection unit 144 detects a glasses candidate region which is a candidate in which glasses worn on the human body are shown as a candidate region from the far-infrared image. In addition, the hair detection unit 145 detects a hair candidate region which is a candidate in which hairs of the human body are shown as a candidate region from the far-infrared image. These detection units detect regions indicating temperatures within mutually different setting temperature ranges from the far-infrared image.

Here, the eye of the human body, the glasses worn on the human body, and the hairs of the human body are equivalent to parts related to a face (hereinafter also referred to as face parts). In addition, the eye candidate region, the glasses candidate region, and the hair candidate region are equivalent to a face part candidate region which is a candidate in which a face part is shown. In addition, the eye detection unit 143, the glasses detection unit 144, and the hair detection unit 145 are equivalent to a face part detection unit that detects a face part candidate region as a candidate region from the far-infrared image.

Each detection unit according to the first application example has functions of the first extraction unit 41a (the second extraction unit 42a), the first score calculation unit 41b (the second score calculation unit 42b), and the first score comparison unit 41c (the second score comparison unit 42c) in the first detection unit 41 (the second detection unit 42) of the image processing device 1 described with reference to FIG. 3. Specifically, each detection unit according to the first application example extracts a partial region from the far-infrared image, calculates a score value of the extracted partial region, and compares the calculated score value to the threshold as in each detection unit of the image processing device 1 described with reference to FIG. 3. In addition, each detection unit according to the first application example detects a corresponding partial region as a candidate region in a case in which the score value is greater than the threshold, and does not detect a corresponding partial region as a candidate region in a case in which the score value is equal to or less than the threshold. In addition, each detection unit according to the first application example outputs a detection result to the determination unit 150.

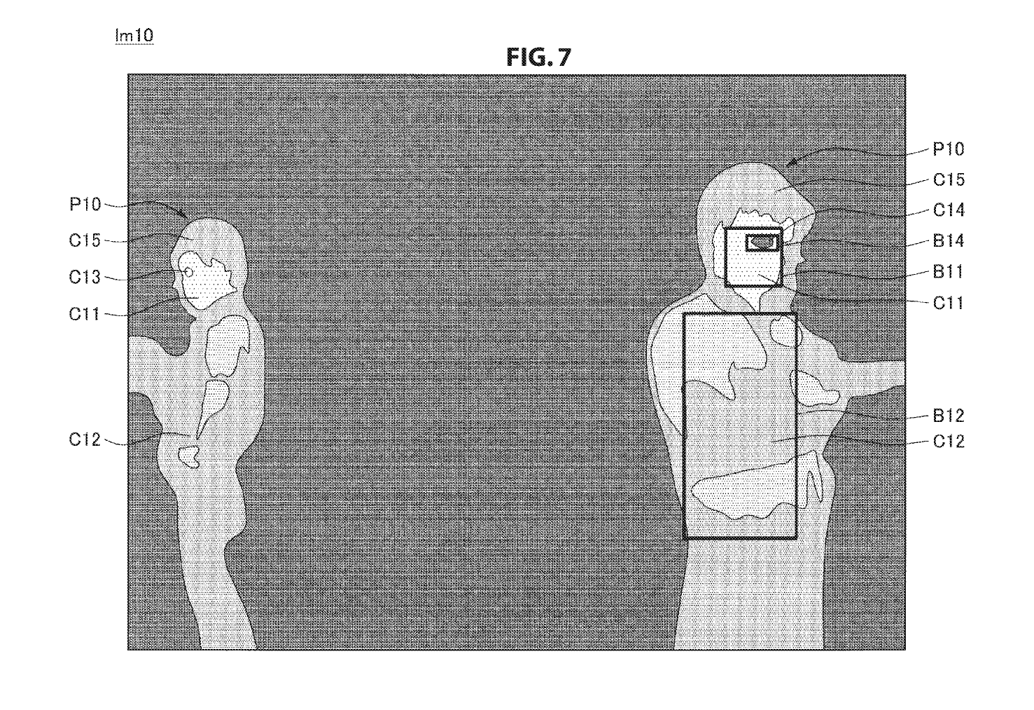

FIG. 7 is an explanatory diagram illustrating an example of a far-infrared image Im10 in which human bodies P10 are shown. As illustrated in FIG. 7, in the far-infrared image Im10, two human bodies P10 are shown as subjects. In addition, in the far-infrared image Im10, a face C11, a trunk C12, glasses C14, and hairs C15 are shown as targets with regard to the right human body P10. In addition, in the far-infrared image Im10, a face C11, a trunk C12, an eye C13, and hairs C15 are shown as targets in the left human body P10. For example, each detection unit according to the first application example can detect candidate regions of candidates in which the targets are shown in the far-infrared image Im10. In the far-infrared image Im10 illustrated in FIG. 7, shade of hatching indicates differences of pixel values. A segment with the deeper hatching is a segment with lower pixel values. In other words, a segment with the deeper hatching is a segment with a lower temperature indicated by the segment.

For example, each detection unit extracts a partial region with predetermined dimensions from the far-infrared image Im10. Specifically, each detection unit can set dimensions of a partial region by referring to the data table D10 stored in the storage unit 160. In the data table D10, for example, as illustrated in FIG. 8, information indicating each target is associated with information indicating dimensions in accordance with each target.

Specifically, the face detection unit 141 sets dimensions “a height of 20 to 30 cm and a width of 15 to 20 cm” corresponding to the face C11 which is a target as dimensions of a partial region. In addition, the trunk detection unit 142 sets dimensions “a height of 50 to 100 cm and a width of 30 to 60 cm” corresponding to the trunk C12 which is a target as dimensions of a partial region. In addition, the eye detection unit 143 sets dimensions “a width of 2 to 4 cm” corresponding to the eye C13 which is a target as dimensions of a partial region. In addition, the glasses detection unit 144 sets dimensions “a width of 15 to 20 cm and a height of 3 to 6 cm” corresponding to the glasses C14 which is a target as dimensions of a partial region. In addition, the hair detection unit 145 sets dimensions “a height of 1 to 15 cm and a width of 15 to 20 cm” corresponding to the hair C15 which is a target as dimensions of a partial region. FIG. 7 schematically illustrates a partial region B11, a partial region B12, and a partial region B14 in which the foregoing dimensions are set to correspond to the face C11, the trunk C12, and the glasses C14, for example.

Then, each detection unit calculates a score value of the partial region on the basis of a probability density function corresponding to an assumed temperature of a target. Specifically, each detection unit can generate the probability density function corresponding to the assumed temperature of the target by referring to the data table D10 stored in the storage unit 160. In the data table D10, for example, as illustrated in FIG. 8, information indicating each target is associated with information indicating the assumed temperature of each target. Note that information indicating the probability density function corresponding to each target may be stored in the storage unit 160. In this case, each detection unit can acquire the information indicating the probability density function from the storage unit 160. In addition, the assumed temperature of each target illustrated in FIG. 8 is, for example, a value in a case in which an environmental temperature is 25° C. The data table may be stored with regard to each environmental temperature in the storage unit 160 or the assumed temperature of each target in each data table can be set in accordance with a corresponding environmental temperature.

Specifically, the face detection unit 141 calculates a score value using “33 to 36° C.” as the assumed temperature of the face C11 which is the target. In addition, the trunk detection unit 142 calculates a score value using “a temperature lower by 2° C. than a face temperature to a temperature lower by 4° C. than the face temperature” as the assumed temperature of the trunk C12 which is the target. In addition, the eye detection unit 143 calculates a score value using “a temperature higher by 1° C. than the face temperature” as the assumed temperature of the eye C13 which is the target. In addition, the glasses detection unit 144 calculates a score value using “a temperature lower by 2° C. than an environmental temperature to a temperature higher by 2° C. than the environmental temperature” as the assumed temperature of the glasses C14 which are the target. In addition, the hair detection unit 145 calculates a score value using “a temperature lower by 3° C. than the face temperature to a temperature lower by 6° C. than the face temperature” as the assumed temperature of the hairs C15 which are the target. In addition, the temperature indicated by the face candidate region detected by the face detection unit 141 can be applied as the face temperature. In addition, a temperature indicated by a region at a predetermined position in the far-infrared image Im10 can be applied as the environmental temperature. The predetermined position may be a position at which there is a relatively high possibility of a background of the human body P10 being shown in the far-infrared image Im10 and may be, for example, an upper end portion of the far-infrared image Im10. Note that the environmental temperature may be acquired using a temperature sensor capable of detecting a temperature of the environment.

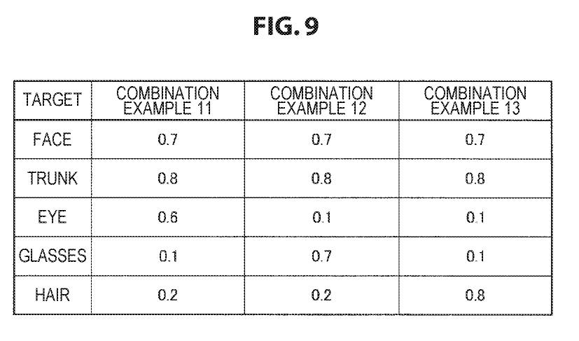

Then, each detection unit compares the calculated score values to the thresholds. The score value takes, for example, a value between 0 and 1. When the score value is larger, a possibility of a temperature indicated by the partial region being the assumed temperature of the target increases. Here, each detection unit repeatedly extracts the partial region so the entire region of the far-infrared image Im10 is scanned, as described above. Therefore, each detection unit calculates the plurality of score values corresponding to the plurality of repeatedly extracted partial regions. An example of a combination of maximum values in the plurality of score values in each detection unit is illustrated in FIG. 9. FIG. 9 illustrates three combination examples.

Specifically, as illustrated in FIG. 9, in combination example 11, maximum values of the score values with regard to the face detection unit 141, the trunk detection unit 142, the eye detection unit 143, the glasses detection unit 144, and the hair detection unit 145 are “0.7,” “0.8,” “0.6,” “0.1”, and “0.2,” respectively. In addition, in combination example 12, maximum values of the score values with regard to the face detection unit 141, the trunk detection unit 142, the eye detection unit 143, the glasses detection unit 144, and the hair detection unit 145 are “0.7,” “0.8,” “0.1,” “0.7”, and “0.2,” respectively. In addition, in combination example 13, maximum values of the score values with regard to the face detection unit 141, the trunk detection unit 142, the eye detection unit 143, the glasses detection unit 144, and the hair detection unit 145 are “0.7,” “0.8,” “0.1,” “0.1”, and “0.8,” respectively.

Here, in a case in which the score value is greater than the threshold, each detection unit detects a corresponding partial region as a candidate region. Conversely, in a case in which the score value is equal to or less than the threshold, each detection unit does not detect the corresponding partial region as the candidate region. Therefore, a case in which the maximum value of the score value with regard to each detection unit is greater than the threshold is equivalent to a case in which each detection unit detects the candidate region. Conversely, a case in which the maximum value of the score value with regard to each detection unit is equal to or less than the threshold is equivalent to a case in which each detection unit does not detect the candidate region.

For example, in a case in which the threshold is set to 0.5, in combination example 11, the face candidate region, the trunk candidate region, and the eye candidate region are detected by the face detection unit 141, the trunk detection unit 142, and the eye detection unit 143, respectively, and the glasses candidate region and the hair candidate region are not detected by the glasses detection unit 144 and the hair detection unit 145, respectively. In addition, in combination example 12, the face candidate region, the trunk candidate region, and the glasses candidate region are detected by the face detection unit 141, the trunk detection unit 142, and the glasses detection unit 144, respectively, and the eye candidate region and the hair candidate region are not detected by the eye detection unit 143 and the hair detection unit 145, respectively. In addition, in combination example 13, the face candidate region, the trunk candidate region, and the hair candidate region are detected by the face detection unit 141, the trunk detection unit 142, and the hair detection unit 145, respectively, and the eye candidate region and the glasses candidate region are not detected by the eye detection unit 143 and the glasses detection unit 144, respectively.

The determination unit 150 according to the first application example determines whether or not the human body P10 is shown as a predetermined subject in the far-infrared image Im10 on the basis of modeling and a positional relation between the detected face candidate region and trunk candidate region. The modeling is an index used to determine whether or not the human body P10 is shown in the far-infrared image Im10. The modeling regulates a positional relation between the face candidate region and the trunk candidate region in a case in which the human body P10 is shown in the far-infrared image Im10.

Specifically, the determination unit 150 determines that the positional relation between the face candidate region and the trunk candidate region is adequate in a case in which the positional relation between the face candidate region and the trunk candidate region is substantially identical to the positional relation regulated by the modeling. Then, the determination unit 150 determines that the human body P10 is shown in the far-infrared image Im10 since the determination unit 150 determines that the positional relation between the face candidate region and the trunk candidate region is adequate.

More specifically, the determination unit 150 can determine whether or not the positional relation between the face candidate region and the trunk candidate region is adequate by referring to the data table D10 stored in the storage unit 160. In the data table D10, for example, as illustrated in FIG. 8, information indicating each target is associated with information indicating a relative position of each object to another object in a case in which the human body P10 is shown in the far-infrared image Im10. The relative position is regulated by the modeling in the first application example.

Specifically, in a case in which the face candidate region is located above the trunk candidate region, the determination unit 150 determines that the positional relation between the face candidate region and the trunk candidate region is adequate. In other words, in a case in which the trunk candidate region is located below the face candidate region, the determination unit 150 determines that the positional relation between the face candidate region and the trunk candidate region is adequate.

In this way, in the first application example, whether or not the human body P10 is shown in the far-infrared image Im10 is determined on the basis of the modeling and the positional relation between the face candidate region and the trunk candidate region indicating the temperatures within mutually different setting temperature ranges. Here, the face C11 and the trunk C12 is present to correspond to the human body P10 and have a positional relation regulated by the modeling. Therefore, when it is determined whether or not the human body P10 is shown in the far-infrared image Im10, a more likely determination result can be obtained. Consequently, detection precision of the human body P10 which is a subject can be improved without using another device different from the infrared camera 102. Accordingly, it is possible to improve the detection precision of the human body P10 which is a subject at a lower cost.

Note that in a case in which the plurality of human bodies P10 are shown as subjects in the far-infrared image as in the far-infrared image Im10 illustrated in FIG. 7, the plurality of face candidate regions or trunk candidate regions can be detected. In this case, the determination unit 150 determines whether or not the positional relation between the face candidate region and the trunk candidate region is adequate, for example, in all of the combinations of the face candidate region and the trunk candidate region. In a case in which the number of combinations between the face candidate region and the trunk candidate region for which the positional relation is determined to be adequate is plural, the determination unit 150 can determine that the human body P10 corresponding to each of the plurality of combinations is shown in the far-infrared image.

In addition, the determination unit 150 may determine whether or not the human body P10 is shown as a predetermined subject in the far-infrared image Im10 on the basis of modeling and a positional relation between the detected face candidate region and the face part candidate region. The modeling regulates a positional relation between the face candidate region and the face part candidate region in a case in which the human body P10 is shown in the far-infrared image Im10.

Specifically, in a case in which the positional relation between the face candidate region and the face part candidate region is substantially identical to a positional relation regulated by the modeling, the determination unit 150 determines that the positional relation between the face candidate region and the face part candidate region is adequate. In addition, the determination unit 150 determines that the human body P10 is shown in the far-infrared image Im10 since the determination unit 150 determines that the positional relation between the face candidate region and the trunk candidate region is adequate and further determines that the positional relation between the face candidate region and the face part candidate region is adequate. For example, the determination unit 150 may determine that the human body P10 is shown in the far-infrared image Im10 in a case in which the determination unit 150 determines that the positional relation between the face candidate region and the trunk candidate region is adequate and determines that the positional relation with at least one face part among the positional relations between the face candidate region and the face part candidate regions is adequate.

More specifically, the determination unit 150 can determine whether or not the positional relation between the face candidate region and the face part candidate region is adequate by referring to the data table D10 stored in the storage unit 160. In the data table D10, for example, as illustrated in FIG. 8, information indicating respective face parts as the targets is associated with information indicating a relative position of each object to another object in a case in which the human body P10 is shown in the far-infrared image Im10. The relative position is regulated by the modeling in the first application example.

Specifically, in a case in which the eye candidate region is located inside the face candidate region, the determination unit 150 determines that the positional relation between the face candidate region and the eye candidate region is adequate. In addition, in a case in which the glasses candidate region is located inside the face candidate region, the determination unit 150 determines that the positional relation between the face candidate region and the glasses candidate region is adequate. In addition, in a case in which the hair candidate region is adjacent above the face candidate region, the determination unit 150 determines that the positional relation between the face candidate region and the hair candidate region is adequate.

Here, the face C11 and each face part is present to correspond to the face C11 and has a positional relation regulated by the modeling. Therefore, in a case in which the positional relation between the face candidate region and the face part candidate region is determined to be adequate on the basis of the modeling, a further more likely determination result can be obtained by determining that the human body P10 is shown in the far-infrared image Im10 when whether or not the human body P10 is shown in the far-infrared image Im10 is determined. Hence, it is possible to more effectively. improve the detection precision of the human body P10 which is a subject.

Note that in a case in which the plurality of human bodies P10 are shown as subjects in a far-infrared image as in the far-infrared image Im10 illustrated in FIG. 7, as described above, the determination unit 150 can determine that the positional relation is adequate in a plurality of combinations of the face candidate region and the trunk candidate region. In this case, for example, the determination unit 150 determines whether or not the positional relation between the face candidate region and the face part candidate region is adequate with regard to each of the plurality of combinations.

In addition, the determination unit 150 may register the determination result by outputting the determination result to the storage unit 160.

In addition, the determination unit 150 may decide whether or not the determination process of determining whether or not the human body P10 is shown in the far-infrared image Im10 is performed in accordance with the detection result output from each detection unit.

For example, in a case in which both the face candidate region and the trunk candidate region are detected, the determination unit 150 may perform the determination process on the positional relation between the face candidate region and the trunk candidate region. Conversely, in a case in which at least one of the face candidate region or the trunk candidate region is not detected, the determination unit 150 may not perform the determination process on the positional relation between the face candidate region and the trunk candidate region.

In addition, in a case in which both the face candidate region and the trunk candidate region are detected and at least one of the face part candidate regions is detected, the determination unit 150 may perform the determination process on the positional relation between the face candidate region and the face part candidate region. Conversely, in a case in which at least one of the face candidate region or the trunk candidate region is not detected or a case in which none of the face part candidate regions is detected, the determination unit 150 may not perform the determination process on the positional relation between the face candidate region and the face part candidate region.

For example, in each of combination examples 11 to 13 illustrated in FIG. 9, both the face candidate region and the trunk candidate region are detected and at least one of the face part candidate regions is detected. Therefore, the determination unit 150 performs the determination process on the positional relation between the face candidate region and the trunk candidate region and the determination process on the positional relation between the face candidate region and the face part candidate region.

The example in which the detection of the candidate regions from the far-infrared image is realized by performing the process of extracting the partial regions, the process of calculating the score values, and the process of comparing the score values to the thresholds has been described above, but a specific method of detecting the candidate regions is not limited to this example.

For example, the image processing device 10 may detect a plurality of candidate regions indicating temperatures within mutually different setting temperature ranges from a far-infrared image by performing image processing such as template matching in accordance with an object on the far-infrared image. In addition, the image processing device 10 may detect a plurality of candidate regions indicating temperatures within mutually different setting temperature ranges from a far-infrared image by using a prediction module learned in advance. The prediction module can be constructed in accordance with a known algorithm such as a boosting or support vector machine, for example, by using a pair of prepared far-infrared image and detection result of a candidate region.

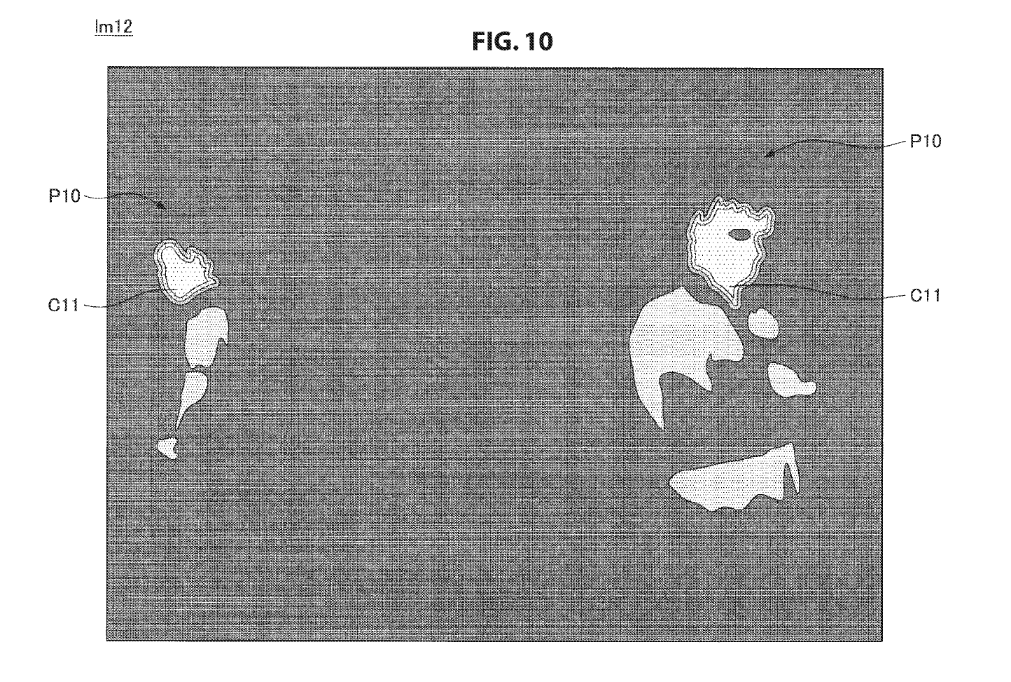

In addition, the image processing device 10 may detect a plurality of candidate regions indicating temperatures within mutually different setting temperature ranges from a far-infrared image by converting a pixel value of each pixel into a likelihood which is an assumed temperature of each target with regard to each pixel of a far-infrared image and performing image processing such as template matching on the converted image. Specifically, the pixel value of each pixel of a far-infrared image can be converted into the likelihood by using the probability density function in accordance with the Gauss distribution of which the middle value is identical to the assumed temperature of each target. Note that in a case in which the assumed temperature has a width, the probability density function in accordance with the Gauss distribution of which the middle value is identical to a middle value of the assumed temperature of each target can be used. FIG. 10 illustrates an image Im12 after the conversion from a pixel value of each pixel into a likelihood. Specifically, the image Im12 illustrated in FIG. 10 is an image obtained by converting the pixel value of each pixel into a likelihood for which a temperature indicated by each pixel is an assumed temperature of a face C11 which is a target with regard to each pixel of the far-infrared image Im10 illustrated in FIG. 7. In the image Im12 illustrated in FIG. 10, shade of hatching indicates differences of the likelihood. A segment with the deeper hatching is a segment with a lower likelihood.

### (Operation)

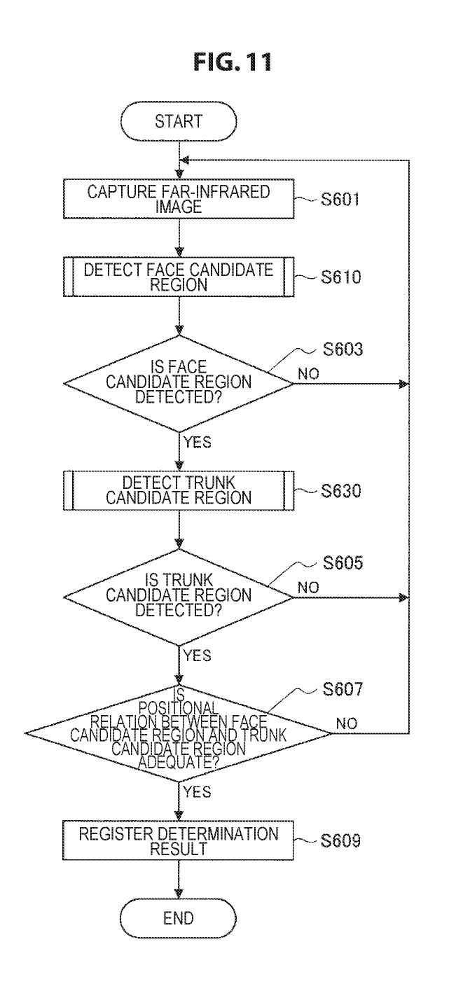

Next, a flow of a process performed by the image processing device 10 according to the first application example will be described with reference to FIGS. 11 to 14.

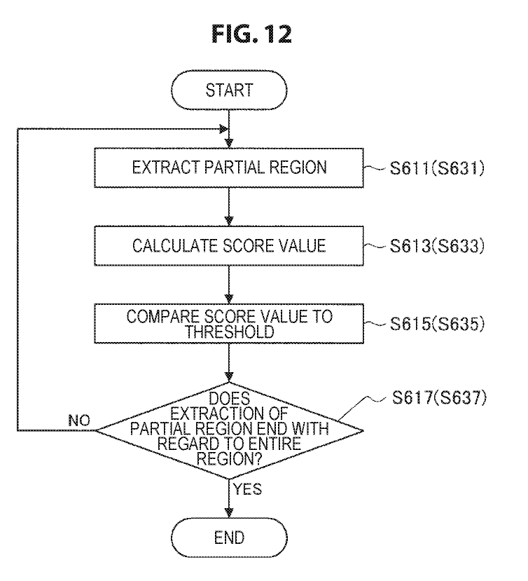

First, a first example of the flow of the process performed by the image processing device 10 according to the first application example will be described with reference to FIGS. 11 and 12. FIG. 11 is a flowchart illustrating the first example of the flow of the process performed by the image processing device 10 according to the first application example. The process illustrated in FIG. 11 can be performed on, for example, each frame.

In the first example, as illustrated in FIG. 11, the image processing device 10 first captures the far-infrared image Im10 (step S601). Subsequently, the face detection unit 141 performs a detection process of detecting the face candidate region from the captured far-infrared image Im10 (step S610) and outputs a detection result to the determination unit 150. Then, the determination unit 150 determines whether or not the face candidate region is detected (step S603). In a case in which it is determined that the face candidate region is not detected (NO in step S603), the process returns to step S601.

Conversely, in a case in which it is determined that the face candidate region is detected (YES in step S603), a determination result is output from the determination unit 150 to the trunk detection unit 142. The trunk detection unit 142 performs a detection process of detecting the trunk candidate region (step S630) and outputs a detection result to the determination unit 150. Then, the determination unit 150 determines whether or not the trunk candidate region is detected (step S605). In a case in which it is determined that the trunk candidate region is not detected (NO in step S605), the process returns to step S601.

Conversely, in a case in which it is determined that the trunk candidate region is detected (YES in step S605), the determination unit 150 determines whether or not the positional relation between the face candidate region and the trunk candidate region is adequate (step S607). In a case in which it is determined that the positional relation between the face candidate region and the trunk candidate region is not substantially identical to the positional relation regulated by the modeling, it is determined that the positional relation between the face candidate region and the trunk candidate region is not adequate (NO in step S607) and the process returns to step S601. Conversely, in a case in which it is determined that the positional relation between the face candidate region and the trunk candidate region is substantially identical to the positional relation regulated by the modeling, it is determined that the positional relation between the face candidate region and the trunk candidate region is adequate (YES in step S607). The determination unit 150 registers a determination result in the storage unit 160 (step S609) and ends the process illustrated in FIG. 11.

Next, the detection processes for the face candidate region and the trunk candidate region (steps S610 and S630 illustrated in FIG. 11) performed by the image processing device 10 according to the first application example will be described in more detail with reference to FIG. 12. FIG. 12 is a flowchart illustrating an example of a flow of a detection process for the face candidate region and the trunk candidate region performed by the image processing device 10 according to the first application example.

As illustrated in FIG. 12, the face detection unit 141 (the trunk detection unit 142) first extracts a partial region from the far-infrared image Im10 (step S611 (step S631)). Subsequently, the face detection unit 141 (the trunk detection unit 142) calculates a score value of the extracted partial region (step S613 (step S633)). Subsequently, the face detection unit 141 (the trunk detection unit 142) compares the calculated score value to the threshold (step S615 (step S635)). Then, the face detection unit 141 (the trunk detection unit 142) determines whether or not the extraction of the partial region ends with regard to the entire region of the far-infrared image Im10 (step S617 (step S637)). In a case in which it is determined that the extraction of the partial region does not end with regard to the entire region of the far-infrared image Im10 (NO in step S617 (NO in step S637)), the process returns to step S611 (step S631). Conversely, in a case in which it is determined that the extraction of the partial region ends with regard to the entire region of the far-infrared image Im10 (YES in step S617 (YES in step S637)), the process illustrated in FIG. 12 ends.

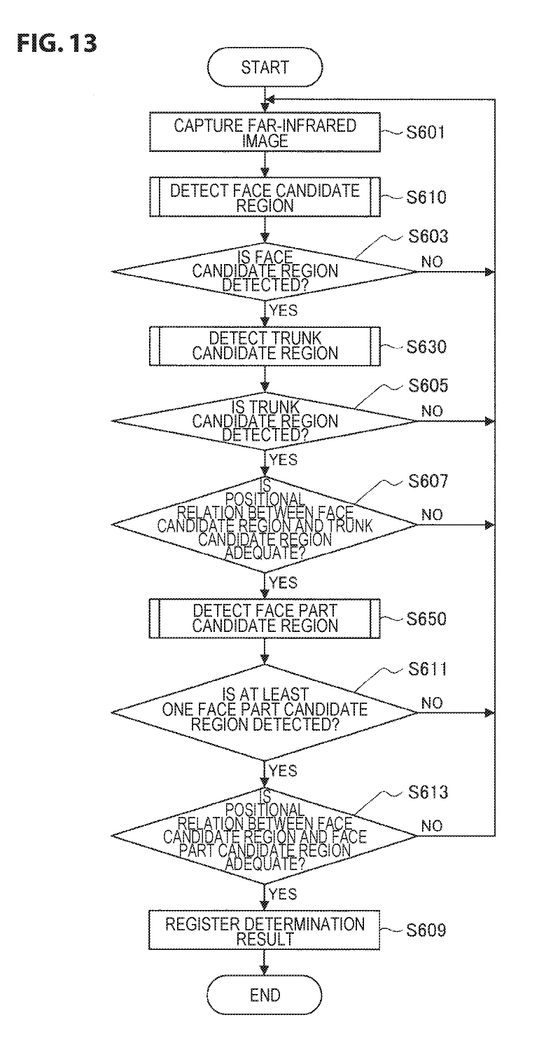

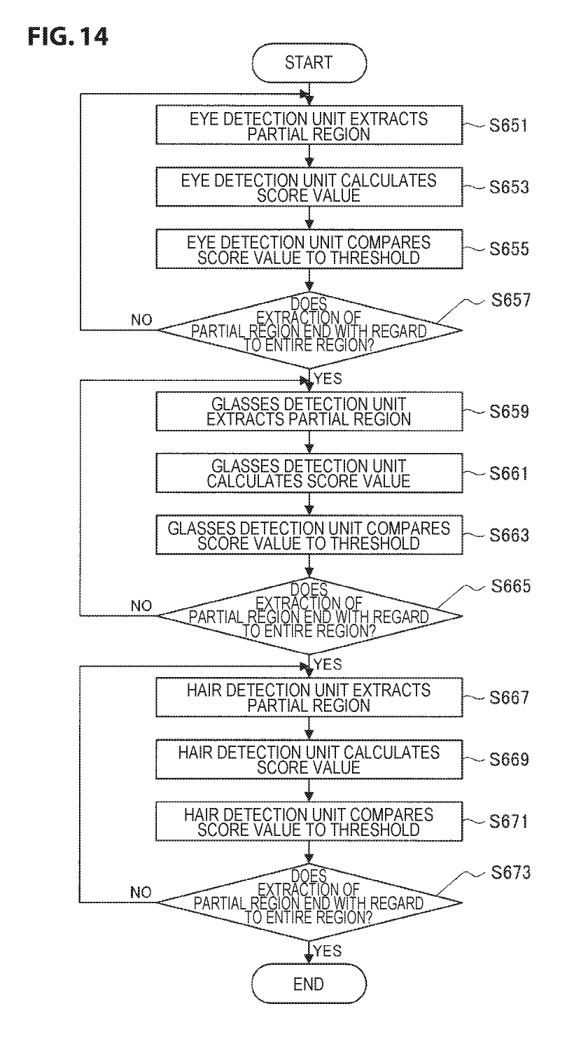

Next, a second example of the flow of the process performed by the image processing device 10 according to the first application example will be described with reference to FIGS. 13 and 14. FIG. 13 is a flowchart illustrating the second example of the flow of the process performed by the image processing device 10 according to the first application example. The second example is different in a process in a case in which it is determined that the positional relation between the face candidate region and the trunk candidate region is adequate (YES in step S607) in the determination process (step S607) for the positional relation, compared to the first example described with reference to FIG. 11. Hereinafter, a flow of the process in the case in which it is determined that the positional relation is adequate (YES in step S607) will be described.

In the second example, as illustrated in FIG. 13, in the case in which it is determined that the positional relation between the face candidate region and the trunk candidate region is adequate in the determination process of step S607 (YES in step S607), a determination result is output from the determination unit 150 to the eye detection unit 143, the glasses detection unit 144, and the hair detection unit 145. Then, the eye detection unit 143, the glasses detection unit 144, and the hair detection unit 145 perform the detection process of detecting the face part candidate region (step S650) and output a detection result to the determination unit 150. Then, the determination unit 150 determines whether or not at least one face part candidate region is detected (step S611). In a case in which it is determined that at least one face part candidate region is not detected (NO in step S611), the process returns to step S601.

Conversely, in a case in which it is determined that at least one face part candidate region is detected (YES in step S611), the determination unit 150 determines whether or not the positional relation between the face candidate region and the face part candidate region is adequate (step S613). In a case in which it is determined that the positional relation between the face candidate region and the face part candidate region is not substantially identical to the positional relation regulated by the modeling, it is determined that the positional relation between the face candidate region and the face part candidate region is not adequate (NO in step S613) and the process returns to step S601. Conversely, in a case in which it is determined that the positional relation between the face candidate region and the face part candidate region is substantially identical to the positional relation regulated by the modeling, it is determined that the positional relation between the face candidate region and the face part candidate region is adequate (YES in step S613). The determination unit 150 registers a determination result in the storage unit 160 (step S609) and ends the process illustrated in FIG. 13.

Next, the detection process (step S650 illustrated in FIG. 13) for the face part candidate region performed by the image processing device 10 according to the first application example will be described in more detail with reference to FIG. 14. FIG. 14 is a flowchart illustrating an example of a flow of a detection process for a face part candidate region performed by the image processing device 10 according to the first application example.

As illustrated in FIG. 14, the eye detection unit 143 first extracts a partial region from the far-infrared image Im10 (step S651). Subsequently, the eye detection unit 143 calculates a score value of the extracted partial region (step S653). Subsequently, the eye detection unit 143 compares the calculated score value to the threshold (step S655). Then, the eye detection unit 143 determines whether or not the extraction of the partial region ends with regard to the entire region of the far-infrared image Im10 (step S657). In a case in which it is determined that the extraction of the partial region does not end with regard to the entire region of the far-infrared image Im10 (NO in step S657), the process returns to step S651.

Conversely, in a case in which it is determined that the extraction of the partial region ends with regard to the entire region of the far-infrared image Im10 (YES in step S657), the glasses detection unit 144 first extracts a partial region from the far-infrared image Im10 (step S659). Subsequently, the glasses detection unit 144 calculates a score value of the extracted partial region (step S653). Subsequently, the glasses detection unit 144 compares the calculated score value to the threshold (step S661). Then, the glasses detection unit 144 determines whether or not the extraction of the partial region ends with regard to the entire region of the far-infrared image Im10 (step S665). In a case in which it is determined that the extraction of the partial region does not end with regard to the entire region of the far-infrared image Im10 (NO in step S665), the process returns to step S659.

Conversely, in a case in which it is determined that the extraction of the partial region ends with regard to the entire region of the far-infrared image Im10 (YES in step S665), the hair detection unit 145 first extracts a partial region from the far-infrared image Im10 (step S667). Subsequently, the hair detection unit 145 calculates a score value of the extracted partial region (step S669). Subsequently, the hair detection unit 145 compares the calculated score value to the threshold (step S671). Then, the hair detection unit 145 determines whether or not the extraction of the partial region ends with regard to the entire region of the far-infrared image Im10 (step S673). In a case in which it is determined that the extraction of the partial region does not end with regard to the entire region of the far-infrared image Im10 (NO in step S673), the process returns to step S667. Conversely, in a case in which it is determined that the extraction of the partial region ends with regard to the entire region of the far-infrared image Im10 (YES in step S673), the process illustrated in FIG. 14 ends.

The example in which the eye detection unit 143, the glasses detection unit 144, the hair detection unit 145 perform the detection process on each candidate region in this order has been described above, but the order of the detection process by each detection unit is not limited to this example. In addition, the detection processes by the detection units may be performed in parallel.

### 3-2. Second Application Example

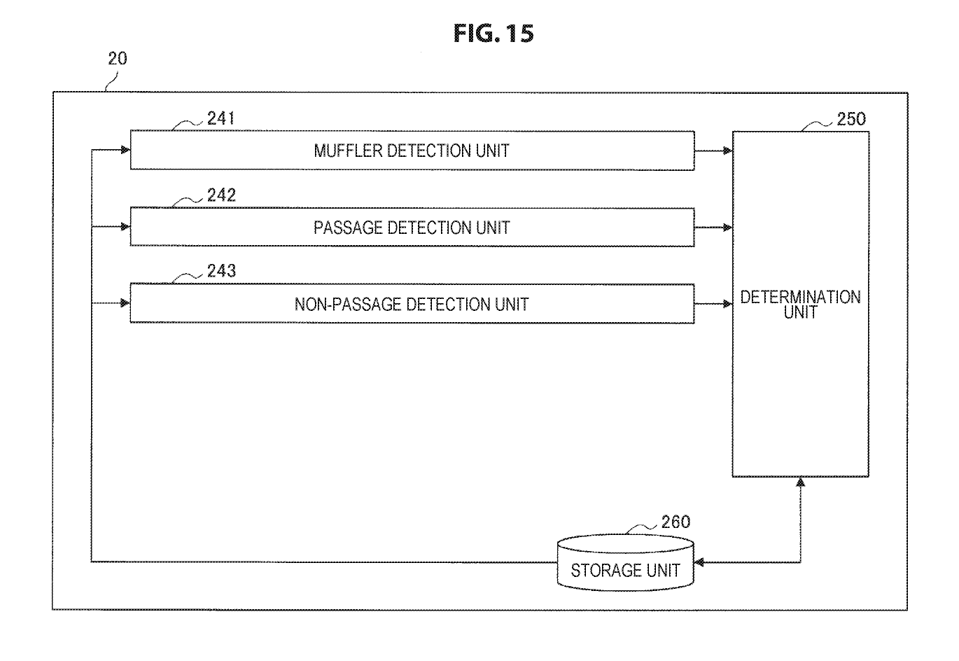

Next, an image processing device 20 according to a second application example will be described with reference to FIGS. 15 to 20. The second application example is an example in which the technology according to the present disclosure is applied to detection of a vehicle which is a subject. The image processing device 20 according to the second application example determines whether or not a vehicle is shown as a predetermined subject in a far-infrared image.

### (Functional Configuration)

First, a functional configuration of the image processing device 20 according to the second application example will be described with reference to FIG. 15. A hardware configuration of the image processing device 20 according to the second application example may be similar to the hardware configuration of the image processing device 1 described with reference to FIG. 2. FIG. 15 is a block diagram illustrating an example of a functional configuration realized by mutually cooperating between constituent elements of the image processing device 20.

As illustrated in FIG. 15, the image processing device 20 includes a muffler detection unit 241, a passage detection unit 242, a non-passage detection unit 243, a determination unit 250, and a storage unit 260. The muffler detection unit 241, the passage detection unit 242, and the non-passage detection unit 243 in the second application example are equivalent to a plurality of detection units according to the present disclosure. In addition, the determination unit 250 and the storage unit 260 in the second application example correspond to the determination unit 50 and the storage unit 60 of the image processing device 1 described with reference to FIG. 3.

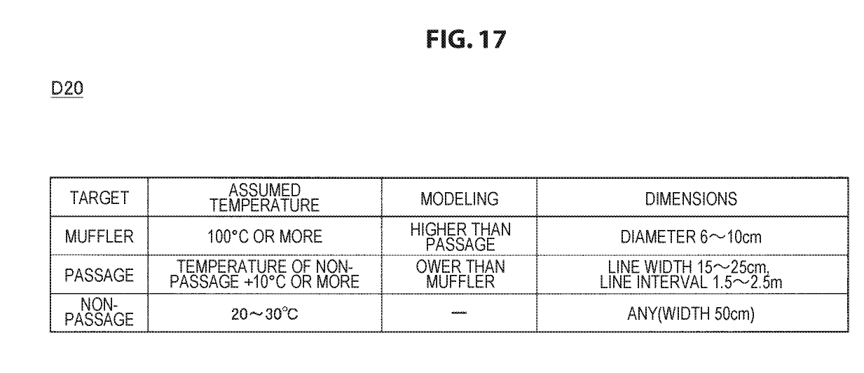

The storage unit 260 stores data which is referred to in each process performed by the image processing device 20. For example, the storage unit 260 stores information which is used in a detection process for the candidate region performed by each of the muffler detection unit 241, the passage detection unit 242, and the non-passage detection unit 243. In addition, the storage unit 260 stores modeling which is used in the determination process performed by the determination unit 250. Specifically, the storage unit 260 stores a data table D20 to be described below with reference to FIG. 17 and various kinds of information are included in the data table D20.

The muffler detection unit 241 detects a muffler candidate region which is a candidate in which a muffler of a vehicle is shown as a candidate region from the far-infrared image. In addition, the passage detection unit 242 detects a passage candidate region which is a candidate in which a portion through which a wheel of the vehicle passes on a road surface is shown as a candidate region from the far-infrared image. In addition, the non-passage detection unit 243 detects a non-passage candidate region which is a candidate in which a portion through which a wheel of the vehicle does not pass on a road surface is shown as a candidate region from the far-infrared image. These detection units detect regions indicating temperatures within mutually different setting temperature ranges from the far-infrared image.

Each detection unit according to the second application example has functions of the first extraction unit 41a (the second extraction unit 42a), the first score calculation unit 41b (the second score calculation unit 42b), and the first score comparison unit 41c (the second score comparison unit 42c) in the first detection unit 41 (the second detection unit 42) of the image processing device 1 described with reference to FIG. 3. Specifically, each detection unit according to the second application example extracts a partial region from the far-infrared image, calculates a score value of the extracted partial region, and compares the calculated score value to the threshold as in each detection unit of the image processing device 1 described with reference to FIG. 3. In addition, each detection unit according to the second application example detects a corresponding partial region as a candidate region in a case in which the score value is greater than the threshold, and does not detect a corresponding partial region as a candidate region in a case in which the score value is equal to or less than the threshold. In addition, each detection unit according to the second application example outputs a detection result to the determination unit 150.

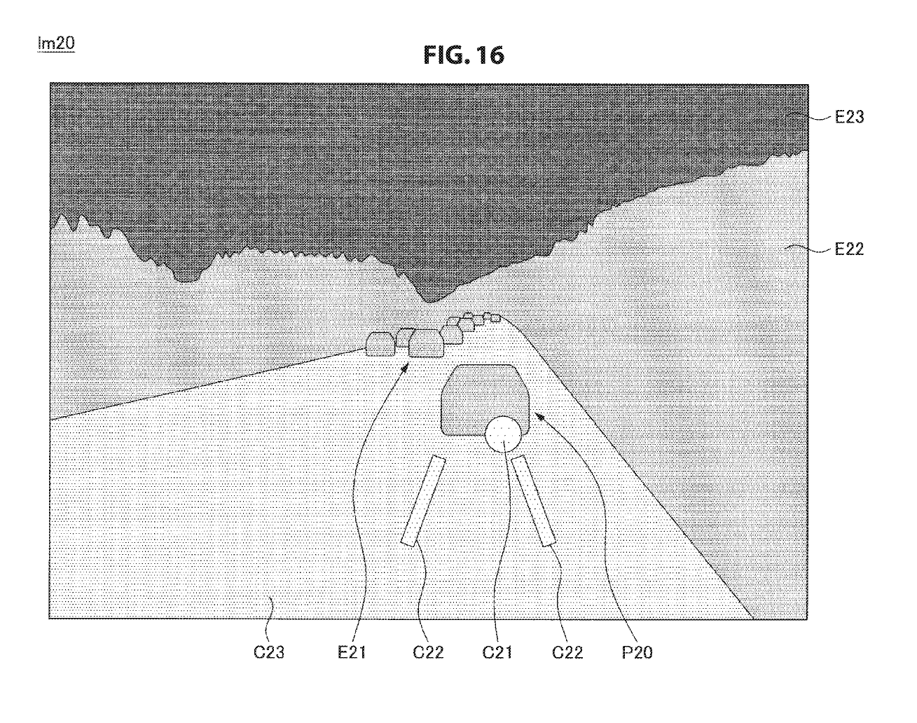

FIG. 16 is an explanatory diagram illustrating an example of a far-infrared image Im20 in which a vehicle P20 is shown. As illustrated in FIG. 16, the vehicle P20 which is a subject travelling on a road surface is shown in the far-infrared image Im20. The image processing device 20 according to the second application example is mounted on, for example, a following vehicle of the vehicle P20 and an infrared camera 102 can be provided on the front side of the following vehicle. In the image processing device 20, the vehicle P20 immediately in front of the vehicle on which the infrared camera 102 is provided is mainly a detection target subject. In addition, in the far-infrared image Im20, a muffler C21 of the vehicle P20, a passage C22 which is a portion through which a wheel of the vehicle on the road surface passes, and a non-passage C23 which is a portion through which a wheel of the vehicle does not passes on the road surface are shown as targets. Each detection unit according to the second application example can detect, for example, a candidate region of a candidate in which a target is shown in the far-infrared image Im20. Note that, as illustrated in FIG. 16, for example, a vehicle group E21 located on the relative front side of the road surface, woods E22 located on both sides of the road surface, and a sky E23 located above are shown in the far-infrared image Im20. In the far-infrared image illustrated in FIG. 16, shade of hatching indicates differences of pixel values. A segment with the deeper hatching is a segment with lower pixel values. In other words, a segment with the deeper hatching is a segment with a lower temperature indicated by the segment.

For example, each detection unit extracts a partial region with predetermined dimensions from the far-infrared image Im20. Specifically, each detection unit can set dimensions of a partial region by referring to the data table D20 stored in the storage unit 260. In the data table D20, for example, as illustrated in FIG. 17, information indicating each target is associated with information indicating dimensions in accordance with each target.

Specifically, the muffler detection unit 241 sets a dimension “a diameter of 6 to 10 cm” corresponding to the muffler C21 which is a target as a dimension of a partial region. In addition, the passage detection unit 242 sets dimensions “a line width of 15 to 25 cm and a line interval of 1.5 to 2.5 m” corresponding to the passage C22 which is a target as dimensions of a partial region. In addition, the non-passage detection unit 243 sets any dimension (for example, a width of 50 cm) as a dimension of the partial region with regard to the non-passage C23 which is a target.

Then, each detection unit calculates a score value of the partial region on the basis of a probability density function corresponding to an assumed temperature of a target. Specifically, each detection unit can generate the probability density function corresponding to the assumed temperature of the target by referring to the data table D20 stored in the storage unit 260. In the data table D20, for example, as illustrated in FIG. 17, information indicating each target is associated with information indicating the assumed temperature of each target. Note that information indicating the probability density function corresponding to each target may be stored in the storage unit 260. In this case, each detection unit can acquire the information indicating the probability density function from the storage unit 260. In addition, the assumed temperature of each target illustrated in FIG. 17 is, for example, a value in a case in which an environmental temperature is 25° C. The data table may be stored with regard to each environmental temperature in the storage unit 260 or the assumed temperature of each target in each data table can be set in accordance with a corresponding environmental temperature.

Specifically, the muffler detection unit 241 calculates a score value using “100° C. or more” as the assumed temperature of the muffler C21 which is the target. In addition, the passage detection unit 242 calculates a score value using “a temperature higher by 10° C. than the temperature of the non-passage” as the assumed temperature of the passage C22 which is the target. In addition, the non-passage detection unit 243 calculates a score value using “20° C. to 30° C.” as the assumed temperature of the non-passage C23 which is the target. Note that the temperature indicated by the non-passage candidate region detected by the non-passage detection unit 243 can be applied as the temperature of the non-passage temperature. In this way, the temperature of the passage C22 is assumed to be higher than that of the non-passage C23.

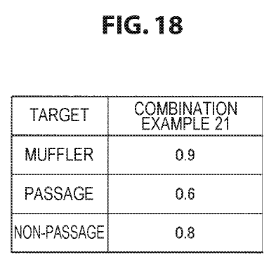

Then, each detection unit compares the calculated score values to the thresholds. The score value takes, for example, a value between 0 and 1. When the score value is larger, a possibility of a temperature indicated by the partial region being the assumed temperature of the target increases. Here, each detection unit repeatedly extracts the partial region so the entire region of the far-infrared image Im20 is scanned, as described above. Therefore, each detection unit calculates the plurality of score values corresponding to the plurality of repeatedly extracted partial regions. Note that the non-passage detection unit 243 may extract the partial region with regard to only a predetermined position in the far-infrared image Im20. The predetermined position is a position at which there is a relatively high possibility of the non-passage being shown in the far-infrared image Im20 and may be, for example, a lower end portion of the far-infrared image Im20. In this case, the non-passage detection unit 243 calculates one score value corresponding to a partial region extracted with regard to the predetermined position. An example of a combination of maximum values of the plurality of score values with regard to the detection units is illustrated in FIG. 18.

Specifically, as illustrated in FIG. 18, in combination example 21, maximum values of the score values with regard to the muffler detection unit 241, the passage detection unit 242, and the non-passage detection unit 243 are “0.9,” “0.6,” and “0.8.”

Here, in a case in which the score value is greater than the threshold, each detection unit detects a corresponding partial region as a candidate region. Conversely, in a case in which the score value is equal to or less than the threshold, each detection unit does not detect the corresponding partial region as the candidate region. Therefore, a case in which the maximum value of the score value with regard to each detection unit is greater than the threshold is equivalent to a case in which each detection unit detects the candidate region. Conversely, a case in which the maximum value of the score value with regard to each detection unit is equal to or less than the threshold is equivalent to a case in which each detection unit does not detect the candidate region.

For example, in a case in which the threshold is set to 0.5, in combination example 21, the muffler candidate region, the passage candidate region, and the non-passage candidate region are detected by the muffler detection unit 241, the passage detection unit 242, and the non-passage detection unit 243, respectively.

The determination unit 250 according to the second application example determines whether or not the vehicle P20 is shown as a predetermined subject in the far-infrared image Im20 on the basis of modeling and a positional relation between the detected muffler candidate region and passage candidate region. The modeling is an index used to determine whether or not the vehicle P20 is shown in the far-infrared image Im20. The modeling regulates a positional relation between the muffler candidate region and the passage candidate region in a case in which the vehicle P20 is shown in the far-infrared image Im20.

Specifically, the determination unit 250 determines that the positional relation between the muffler candidate region and the passage candidate region is adequate in a case in which the positional relation between the muffler candidate region and the passage candidate region is substantially identical to the positional relation regulated by the modeling. Then, the determination unit 250 determines that the vehicle P20 is shown in the far-infrared image Im20 since the determination unit 250 determines that the positional relation between the muffler candidate region and the passage candidate region is adequate.

More specifically, the determination unit 250 can determine whether or not the positional relation between the muffler candidate region and the passage candidate region is adequate by referring to the data table D20 stored in the storage unit 260. In the data table D10, for example, as illustrated in FIG. 17, information indicating each target is associated with information indicating a relative position of each object to another object in a case in which the vehicle P20 is shown in the far-infrared image Im20. The relative position is regulated by the modeling in the second application example. Note that since the non-passage candidate region is basically detected to calculate the assumed temperature of the passage C22, a relative position of the non-passage C23 with respect to another target may not be regulated in the data table D20, as illustrated in FIG. 17.

Specifically, in a case in which the muffler candidate region is located above the passage candidate region, the determination unit 250 determines that the positional relation between the muffler candidate region and the passage candidate region is adequate. In other words, in a case in which the passage candidate region is located below the muffler candidate region, the determination unit 250 determines that the positional relation between the muffler candidate region and the passage candidate region is adequate.

In this way, in the second application example, whether or not the vehicle P20 is shown in the far-infrared image Im20 is determined on the basis of the modeling and the positional relation between the muffler candidate region and the passage candidate region indicating the temperatures within mutually different setting temperature ranges. Here, the muffler C21 and the passage C22 is present to correspond to the vehicle P20 and have a positional relation regulated by the modeling. Therefore, when it is determined whether or not the vehicle P20 is shown in the far-infrared image Im20, a more likely determination result can be obtained. Consequently, detection precision of the vehicle P20 which is a subject can be improved without using another device different from the infrared camera 102. Accordingly, it is possible to improve the detection precision of the vehicle P20 which is a subject at a lower cost.

Note that in a case in which the plurality of vehicle P20 are shown as subjects in the far-infrared image, the plurality of muffler candidate regions or passage candidate regions can be detected. In this case, the determination unit 250 determines whether or not the positional relation between the muffler candidate region and the passage candidate region is adequate, for example, in all of the combinations of the muffler candidate region and the passage candidate region. In a case in which the number of combinations between the muffler candidate region and the passage candidate region for which the positional relation is determined to be adequate is plural, the determination unit 250 can determine that the vehicle P20 corresponding to each of the plurality of combinations is shown in the far-infrared image.

In addition, the determination unit 250 may register the determination result by outputting the determination result to the storage unit 260.

In addition, the determination unit 250 may decide whether or not the determination process of determining whether or not the vehicle P20 is shown in the far-infrared image Im20 is performed in accordance with the detection result output from each detection unit.

For example, in a case in which all the muffler candidate region, the passage candidate region, and the non-passage candidate region are detected, the determination unit 250 may perform the determination process on the positional relation between the muffler candidate region and the passage candidate region. Conversely, in a case in which at least one of the muffler candidate region, the passage candidate region, or the non-passage candidate region is not detected, the determination unit 250 may not perform the determination process on the positional relation between the muffler candidate region and the passage candidate region.

For example, in combination example 21 illustrated in FIG. 18, all the muffler candidate region, the passage candidate region, and the non-passage candidate region are detected. Therefore, the determination unit 250 performs the determination process on the positional relation between the muffler candidate region and the passage candidate region.

The example in which the detection of the candidate regions from the far-infrared image is realized by performing the process of extracting the partial regions, the process of calculating the score values, and the process of comparing the score values to the thresholds has been described above, but a specific method of detecting the candidate regions is not limited to this example as in the first application example. For example, in the second application example, a muffler candidate region can be considered to be detected by performing template matching using a circular or elliptical shape as the shape of the muffler C21. In addition, a passage candidate region can be considered to be detected by performing template matching using a pair of right and left line segment shapes inclined to be more distant on the lower side as the shape of the passage C22.

In addition, the example in which the muffler candidate region and the passage candidate region are detected and the vehicle P20 is detected on the basis of the modeling and the positional relation between the muffler candidate region and the passage candidate region has been described above, the detection of the vehicle P20 may be realized by detecting candidate regions of other targets. For example, a candidate region of a backlight or a tire of the vehicle P20 which is a target can be detected, and the vehicle P20 can be detected on the basis of modeling and a positional relation between the candidate region and another candidate region. As a combination of the candidate regions, for example, various combinations such as a combination of a backlight candidate region which is a candidate region of a backlight and a tire candidate region which is a candidate region of a tire or a combination of a tire candidate region and a passage candidate region can be applied. Note that information indicating a temperature in accordance with a kind of backlight (for example, a halogen light, a light-emitting diode (LED), or the like) can be stored as an assumed temperature of the backlight in the storage unit 260.

### (Operation)

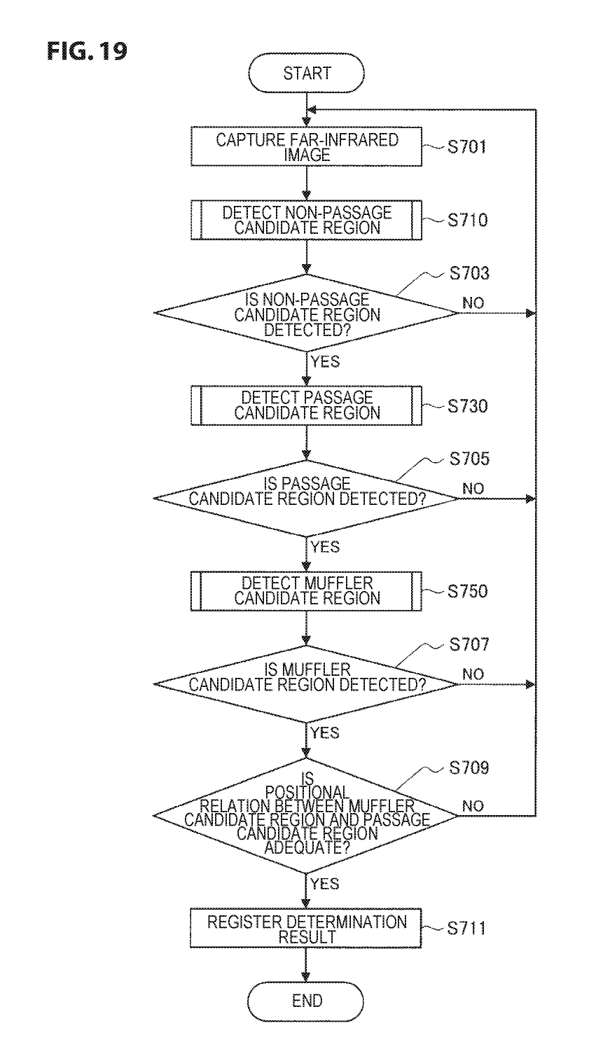

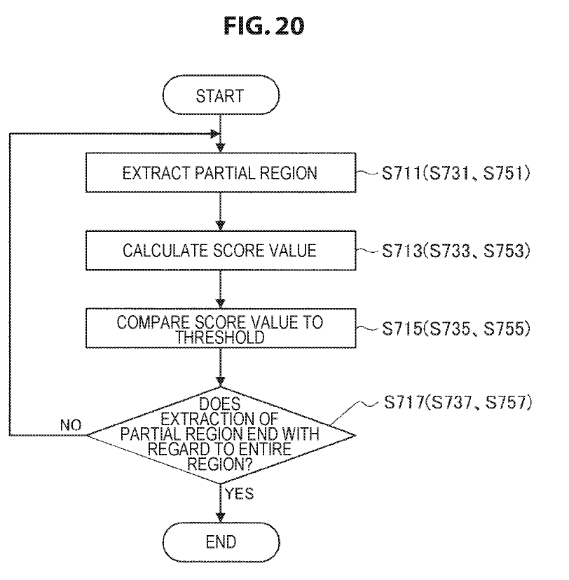

Next, the flow of the process performed by the image processing device 20 according to the second application example will be described with reference to FIGS. 19 and 20. FIG. 19 is a flowchart illustrating an example of the flow of the process performed by the image processing device 20 according to the second application example. The process illustrated in FIG. 19 can be performed on, for example, each frame.

As illustrated in FIG. 19, the image processing device 20 first captures the far-infrared image Im20 (step S701). Subsequently, the non-passage detection unit 243 performs a detection process of detecting the non-passage candidate region from the captured far-infrared image Im20 (step S710) and outputs a detection result to the determination unit 250. Then, the determination unit 250 determines whether or not the non-passage candidate region is detected (step S703). In a case in which it is determined that the non-passage candidate region is not detected (NO in step S703), the process returns to step S701.

Conversely, in a case in which it is determined that the non-passage candidate region is detected (YES in step S703), a determination result is output from the determination unit 250 to the passage detection unit 242. The passage detection unit 242 performs a detection process of detecting the passage candidate region (step S730) and outputs a detection result to the determination unit 250. Then, the determination unit 250 determines whether or not the passage candidate region is detected (step S705). In a case in which it is determined that the passage candidate region is not detected (NO in step S705), the process returns to step S701.

Conversely, in a case in which it is determined that the non-passage candidate region is detected (YES in step S705), a determination result is output from the determination unit 250 to the muffler detection unit 241. The muffler detection unit 241 performs a detection process of detecting the muffler candidate region (step S750) and outputs a detection result to the determination unit 250. Then, the determination unit 250 determines whether or not the muffler candidate region is detected (step S707). In a case in which it is determined that the muffler candidate region is not detected (NO in step S707), the process returns to step S701.

Conversely, in a case in which it is determined that the muffler candidate region is detected (YES in step S707), the determination unit 250 determines whether or not the positional relation between the muffler candidate region and the passage candidate region is adequate (step S709). In a case in which it is determined that the positional relation between the muffler candidate region and the passage candidate region is not substantially identical to the positional relation regulated by the modeling, it is determined that the positional relation between the muffler candidate region and the passage candidate region is not adequate (NO in step S709) and the process returns to step S701. Conversely, in a case in which it is determined that the positional relation between the muffler candidate region and the passage candidate region is substantially identical to the positional relation regulated by the modeling, it is determined that the positional relation between the muffler candidate region and the passage candidate region is adequate (YES in step S709). The determination unit 250 registers a determination result in the storage unit 260 (step S711) and ends the process illustrated in FIG. 19.

Note that an order of the detection process (step S730) for the passage candidate region and the detection process (step S750) for the muffler candidate region is not limited to this example. In addition, the detection process (step S730) for the passage candidate region and the detection process (step S750) for the muffler candidate region may be performed in parallel.

Next, the detection processes for the non-passage face candidate region, the passage candidate region, and the muffler candidate region (steps S710, S730, and S750 illustrated in FIG. 19) performed by the image processing device 20 according to the second application example will be described in more detail with reference to FIG. 20. FIG. 20 is a flowchart illustrating an example of a flow of a detection process for the non-passage face candidate region, the passage candidate region, and the muffler candidate region performed by the image processing device 20 according to the second application example.

As illustrated in FIG. 20, the non-passage detection unit 243 (the passage detection unit 242 and the muffler detection unit 241) first extracts a partial region from the far-infrared image Im20 (step S711 (steps S731 and S751)). Subsequently, the non-passage detection unit 243 (the passage detection unit 242 and the muffler detection unit 241) calculates a score value of the extracted partial region (step S713 (steps S733 and S753)). Subsequently, the non-passage detection unit 243 (the passage detection unit 242 and the muffler detection unit 241) compares the calculated score value to the threshold (step S715 (steps S735 and S755)). Then, the non-passage detection unit 243 (the passage detection unit 242 and the muffler detection unit 241) determines whether the extraction of the partial region ends with regard to the entire region of the far-infrared image Im20 (step S717 (steps S737 and S757)). In a case in which it is determined that the extraction of the partial region does not end with regard to the entire region of the far-infrared image Im20 (NO in step S717 (NO in step S737 and NO in step S757)), the process returns to step S711 (steps S731 and S735). Conversely, in a case in which it is determined that the extraction of the partial region ends with regard to the entire region of the far-infrared image Im20 (YES in step S617 (YES in step S737 and YES in step S757)), the process illustrated in FIG. 20 ends.

Note that, as described above, the non-passage detection unit 243 may extract the partial region with regard to only a predetermined position in the far-infrared image Im20. In this case, the process (step S717) of determining whether or not the extraction of the partial region ends with regard to the entire region of the far-infrared image Im20 is omitted from the flow of the detection process for the non-passage candidate region by the non-passage detection unit 243.

### 3-3. Third Application Example

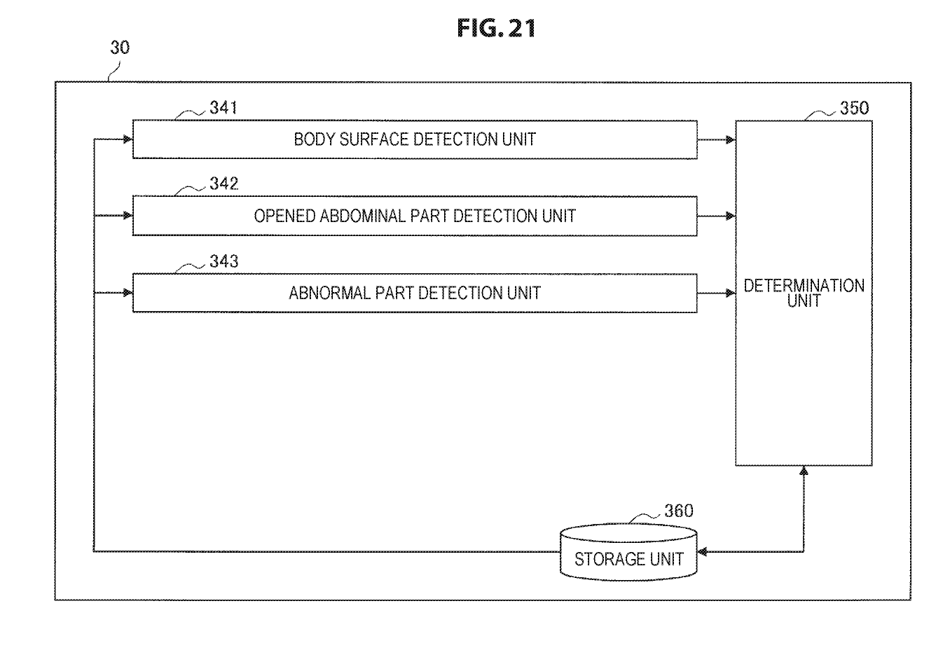

Next, an image processing device 30 according to a third application example will be described with reference to FIGS. 21 to 27. The third application example is an example in which the technology according to the present disclosure is applied to detection of an abnormal part which is a diseased part in an abnormal state in an opened abdominal part of a patient that is a subject. Note that the opened abdominal part is an example of an incised part and the technology according to the present disclosure is also applied to, for example, detection of an abnormal part which is a diseased part in an abnormal state in an opened chest of a patient that is a subject. The image processing device 30 according to the third application example determines whether or not an abnormal part is shown as a predetermined subject in a far-infrared image.

### (Functional Configuration)

First, a functional configuration of the image processing device 30 according to the third application example will be described with reference to FIG. 21. A hardware configuration of the image processing device 30 according to the third application example may be similar to the hardware configuration of the image processing device 1 described with reference to FIG. 2. FIG. 21 is a block diagram illustrating an example of a functional configuration realized by mutually cooperating between constituent elements of the image processing device 30.

As illustrated in FIG. 21, the image processing device 30 includes a body surface detection unit 341, an opened abdominal part detection unit 342, an abnormal part detection unit 343, a determination unit 350, and a storage unit 360. The body surface detection unit 341, the opened abdominal part detection unit 342, and the abnormal part detection unit 343 in the third application example are equivalent to a plurality of detection units according to the present disclosure. In addition, the determination unit 350 and the storage unit 360 in the third application example correspond to the determination unit 50 and the storage unit 60 of the image processing device 1 described with reference to FIG. 3.

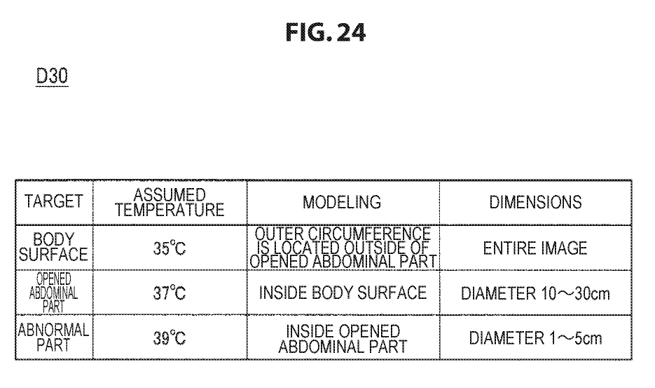

The storage unit 360 stores data which is referred to in each process performed by the image processing device 30. For example, the storage unit 360 stores information which is used in a detection process for the candidate region performed by each of the body surface detection unit 341, the opened abdominal part detection unit 342, and the abnormal part detection unit 343. In addition, the storage unit 360 stores modeling which is used in the determination process performed by the determination unit 350. Specifically, the storage unit 360 stores a data table D30 to be described below with reference to FIG. 24 and various kinds of information are included in the data table D30.

The body surface detection unit 341 detects a body surface candidate region in which a body surface of the patient is shown as a candidate region from the far-infrared image. In addition, the opened abdominal part detection unit 342 detects an opened abdominal part candidate region which is a candidate in which the opened abdominal part of the patient is shown as a candidate region from the far-infrared image. In addition, the abnormal part detection unit 343 detects an abnormal part candidate region which is a candidate in which an abnormal part in the opened abdominal part is shown as a candidate region from the far-infrared image. These detection units detect the regions indicating temperatures within mutually different setting temperature ranges from the far-infrared image.

Here, the opened abdominal part is an example of an incised part, as described above. In addition, the opened abdominal part candidate region is equivalent to an incised part candidate region which is a candidate in which an incised part is shown. In addition, the opened abdominal part detection unit 342 is equivalent to an incised part detection unit that detects an incised part candidate region as a candidate region from the far-infrared image.

Each detection unit according to the third application example has functions of the first extraction unit 41a (the second extraction unit 42a), the first score calculation unit 41b (the second score calculation unit 42b), and the first score comparison unit 41c (the second score comparison unit 42c) in the first detection unit 41 (the second detection unit 42) of the image processing device 1 described with reference to FIG. 3. Specifically, each detection unit according to the third application example extracts a partial region from the far-infrared image, calculates a score value of the extracted partial region, and compares the calculated score value to the threshold as in each detection unit of the image processing device 1 described with reference to FIG. 3. In addition, each detection unit according to the third application example detects a corresponding partial region as a candidate region in a case in which the score value is greater than the threshold, and does not detect a corresponding partial region as a candidate region in a case in which the score value is equal to or less than the threshold. In addition, each detection unit according to the third application example outputs a detection result to the determination unit 350.

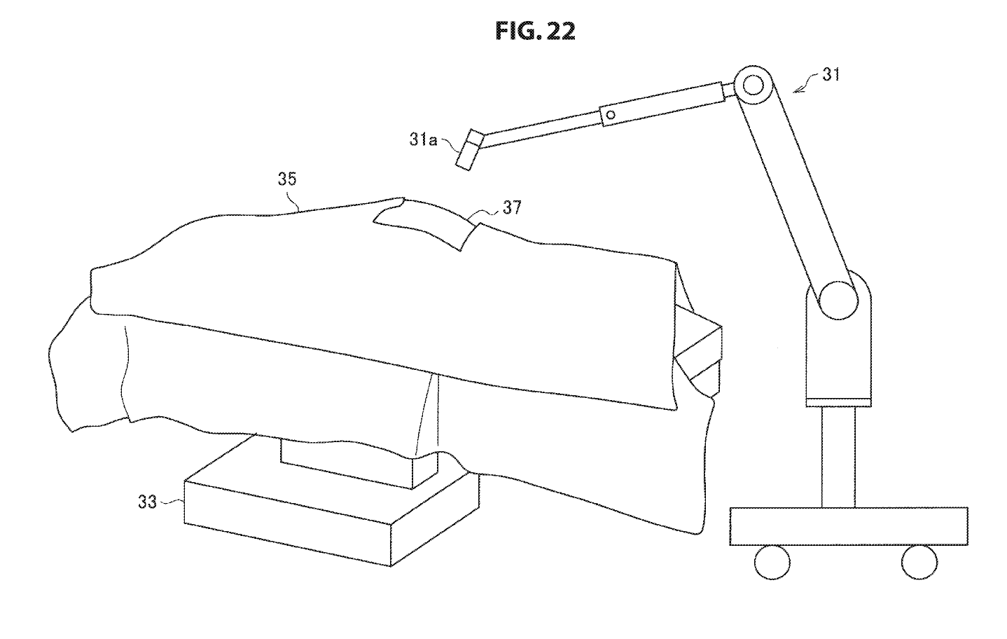

The image processing device 30 according to the third application example is applied to a microscopic device used for so-called microsurgery performed while a minute part of a patient is expanded and observed. FIG. 22 is an explanatory diagram illustrating a surgery in which a microscopic device 31 is used. FIG. 22 schematically illustrates a form in which a surgery part of a patient 37 on a patient bed 33 is imaged by the microscopic device 31 in a surgery operation. Parts except for the surgery part of the patient 37 are covered with an nonwoven fabric 35. In addition, as illustrated in FIG. 22, a camera 31a is provided at the tip end of the microscopic device 31 so that the camera 31a images the surgery part of the patient 37. The microscopic device 31 includes a plurality of arms that are rotatable and can adjust a position and an attitude of the camera 31a by causing the plurality of arms to be appropriately rotated. The microscopic device 31 is connected to a display device (not illustrated), an image of the surgery part is shown on the display device, and thus a surgery operator can perform surgery while checking the image. The microscopic device 31 includes a control device (not illustrated). The image processing device 30 according to the third application example can be applied to, for example, the microscopic device 31. In this case, the camera 31a is equivalent to the infrared camera 102.

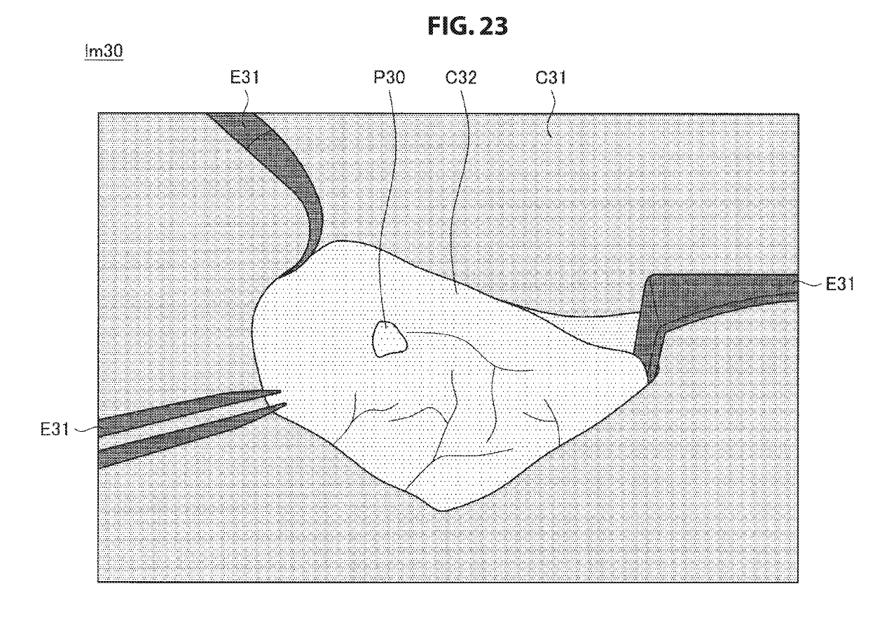

FIG. 23 is an explanatory diagram illustrating an example of a far-infrared image Im30 in which an abnormal part P30 in an opened abdominal part C32 of a patient is shown. As illustrated in FIG. 23, the abnormal part P30 which is a subject is shown in the far-infrared image Im30. In addition, a body surface C31 and an opened abdominal part C32 of the patient that are targets are shown in the far-infrared image Im30. In addition, the abnormal part P30 in the opened abdominal part C32 is equivalent to a detection target subject and is also equivalent to a target in detection of the candidate region by the abnormal part detection unit 343. For example, each detection unit according to the third application example can detect a candidate region of a candidate in which the target is shown in the far-infrared image Im30. Note that, as illustrated in FIG. 23, for example, a plurality of instruments E31 such as forceps supporting the edge of the opened abdominal part C32 are shown in the far-infrared image Im30. In the far-infrared image illustrated in FIG. 23, shade of hatching indicates differences of pixel values. A segment with the deeper hatching is a segment with lower pixel values. In other words, a segment with the deeper hatching is a segment with a lower temperature indicated by the segment.

For example, each detection unit extracts a partial region with predetermined dimensions from the far-infrared image Im30. Specifically, each detection unit can set dimensions of a partial region by referring to the data table D30 stored in the storage unit 360. In the data table D30, for example, as illustrated in FIG. 24, information indicating each target is associated with information indicating dimensions in accordance with each target.

Specifically, the body surface detection unit 341 sets the entire image as dimensions of a partial region with regard to the body surface C31 which is the target. In addition, the opened abdominal part detection unit 342 sets a dimension “a diameter of 10 to 30 cm” corresponding to the opened abdominal part C32 which is the target as a dimension of a partial region. In addition, the abnormal part detection unit 343 sets a dimension “a diameter of 1 to 5 cm” corresponding to the abnormal part P30 which is the target as a dimension of a partial region.

Then, each detection unit calculates a score value of the partial region on the basis of a probability density function corresponding to an assumed temperature of a target. Specifically, each detection unit can generate the probability density function corresponding to the assumed temperature of the target by referring to the data table D30 stored in the storage unit 360. In the data table D30, for example, as illustrated in FIG. 24, information indicating each target is associated with information indicating the assumed temperature of each target. Note that information indicating the probability density function corresponding to each target may be stored in the storage unit 360. In this case, each detection unit can acquire the information indicating the probability density function from the storage unit 360. In addition, in a way similar to the first application example and the second application example, the data table may be stored with regard to each environmental temperature in the storage unit 360 or the assumed temperature of each target in each data table can be set in accordance with a corresponding environmental temperature.

Specifically, the body surface detection unit 341 calculates a score value using “35° C.” as the assumed temperature of the body surface C31 which is the target. In addition, the opened abdominal part detection unit 342 calculates a score value using “37° C.” as the assumed temperature of the opened abdominal part C32 which is the target. In addition, the abnormal part detection unit 343 calculates a score value using “39° C.” as the assumed temperature of the abnormal part P30 which is the target. Since swelling or bleeding occurs in the abnormal part P30 in some cases, the temperature of the abnormal part P30 is assumed to be higher than that of the opened abdominal part C32 in this way.

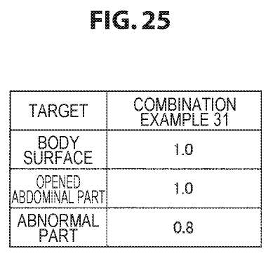

Then, each detection unit compares the calculated score values to the thresholds. The score value takes, for example, a value between 0 and 1. When the score value is larger, a possibility of a temperature indicated by the partial region being the assumed temperature of the target increases. Here, each detection unit repeatedly extracts the partial region so the entire region of the far-infrared image Im30 is scanned, as described above. Therefore, the opened abdominal part detection unit 342 and the abnormal part detection unit 343 calculate a plurality of score values corresponding to a plurality of repeatedly extracted partial regions. Note that the body surface detection unit 341 does not extract the partial region of the far-infrared image Im30 a plurality of times in a case in which the entire image is set as dimensions of the partial region, as described above. An example of a combination of maximum values of the plurality of score values with regard to the opened abdominal part detection unit 342 and abnormal part detection unit 343 and a score value with regard to the body surface detection unit 341 is illustrated in FIG. 25.

Specifically, as illustrated in FIG. 25, in combination example 31, maximum values of the score values with regard to the opened abdominal part detection unit 342 and the abnormal part detection unit 343 are “1.0” and “1.0,” respectively, and the score value with regard to the body surface detection unit 341 is “0.8.”.

Here, in a case in which the score value is greater than the threshold, each detection unit detects a corresponding partial region as a candidate region. Conversely, in a case in which the score value is equal to or less than the threshold, each detection unit does not detect the corresponding partial region as the candidate region. Therefore, a case in which the maximum value of the score value with regard to each of the opened abdominal part detection unit 342 and the abnormal part detection unit 343 is greater than the threshold is equivalent to a case in which each of the opened abdominal part detection unit 342 and the abnormal part detection unit 343 detects the candidate region. Conversely, a case in which the maximum value of the score value with regard to each of the opened abdominal part detection unit 342 and the abnormal part detection unit 343 is equal to or less than the threshold is equivalent to a case in which each of the opened abdominal part detection unit 342 and the abnormal part detection unit 343 does not detect the candidate region.

For example, in a case in which the threshold is set to 0.5, in combination example 31, the body surface candidate region, the opened abdominal part candidate region, and the abnormal part candidate region are detected by the body surface detection unit 341, the opened abdominal part detection unit 342, and abnormal part detection unit 343, respectively.

The determination unit 350 according to the third application example determines whether or not the abnormal part P30 is shown as a predetermined subject in the far-infrared image Im30 on the basis of modeling and a positional relation between the detected body surface candidate region, opened abdominal part candidate region, and abnormal part candidate region. The modeling is an index used to determine whether or not the vehicle P30 is shown in the far-infrared image Im30. The modeling regulates a positional relation between the body surface candidate region, opened abdominal part candidate region, and abnormal part candidate region in a case in which the abnormal part P30 is shown in the far-infrared image Im30.

Specifically, the determination unit 350 determines that the positional relation among the body surface candidate region, opened abdominal part candidate region, and abnormal part candidate region is adequate in a case in which the positional relation among the body surface candidate region, opened abdominal part candidate region, and abnormal part candidate region is substantially identical to the positional relation regulated by the modeling. Then, the determination unit 350 determines that the abnormal part P30 is shown in the far-infrared image Im30 since the determination unit 350 determines that the positional relation among the body surface candidate region, opened abdominal part candidate region, and abnormal part candidate region is adequate.

More specifically, the determination unit 350 can determine whether or not the positional relation among the body surface candidate region, opened abdominal part candidate region, and abnormal part candidate region is adequate by referring to the data table D30 stored in the storage unit 360. In the data table D30, for example, as illustrated in FIG. 24, information indicating each target is associated with information indicating a relative position of each object to another object in a case in which the abnormal part P30 is shown in the far-infrared image Im30. The relative position is regulated by the modeling in the third application example.

Specifically, in a case in which an outer circumference of the body surface candidate region is located outside of the opened abdominal part candidate region and the abnormal part candidate region is located inside the opened abdominal part candidate region, the determination unit 350 determines that a positional relation among the body surface candidate region, the opened abdominal part candidate region, and the abnormal part candidate region is adequate. In other words, in a case in which the opened abdominal part candidate region is located inside the body surface candidate region and the abnormal part candidate region is located inside the opened abdominal part candidate region, the determination unit 350 determines that the positional relation among the positional relation among the body surface candidate region, the opened abdominal part candidate region, and the abnormal part candidate region is adequate.

In this way, in the third application example, whether or not the abnormal part P30 is shown in the far-infrared image Im30 is determined on the basis of the modeling and the positional relation among the body surface candidate region, opened abdominal part candidate region, and abnormal part candidate region indicating the temperatures within mutually different setting temperature ranges. Here, the body surface C31, the opened abdominal part C32, and the abnormal part P30 are present to correspond to the abnormal part P30 and have a positional relation regulated by the modeling. Therefore, when it is determined whether or not the abnormal part P30 is shown in the far-infrared image Im30, a more likely determination result can be obtained. Consequently, detection precision of the abnormal part P30 which is a subject can be improved without using another device different from the infrared camera 102. Accordingly, it is possible to improve the detection precision of the abnormal part P30 which is a subject at a lower cost.

Note that in a case in which the plurality of abnormal parts P30 are shown as subjects in the far-infrared image, the plurality of body surface candidate regions, opened abdominal part candidate regions, or abnormal part candidate regions can be detected. In this case, the determination unit 350 determines whether or not the positional relation among the body surface candidate region, opened abdominal part candidate region, and abnormal part candidate region is adequate, for example, in all of the combinations of the body surface candidate region, opened abdominal part candidate region, and abnormal part candidate region. In a case in which the number of combinations among the body surface candidate region, opened abdominal part candidate region, and abnormal part candidate region for which the positional relation is determined to be adequate is plural, the determination unit 350 can determine that the abnormal part P30 corresponding to each of the plurality of combinations is shown in the far-infrared image.

In addition, the determination unit 350 reports a determination result by outputting the determination result to the display 108. Thus, for example, the surgery operator is warned.

In addition, the determination unit 350 may decide whether or not the determination process of determining whether or not the vehicle P30 is shown in the far-infrared image Im30 is performed in accordance with the detection result output from each detection unit.

For example, in a case in which all the body surface candidate region, the opened abdominal part candidate region, and the abnormal part candidate region are detected, the determination unit 350 may perform the determination process on the positional relation among the body surface candidate region, the opened abdominal part candidate region, and the abnormal part candidate region. Conversely, in a case in which at least one of the body surface candidate region, the opened abdominal part candidate region, or the abnormal part candidate region is not detected, the determination unit 350 may not perform the determination process on the positional relation among the body surface candidate region, the opened abdominal part candidate region, and the abnormal part candidate region.

For example, in combination example 31 illustrated in FIG. 25, all the body surface candidate region, the opened abdominal part candidate region, and the abnormal part candidate region are detected. Therefore, the determination unit 350 performs the determination process on the positional relation among the body surface candidate region, the opened abdominal part candidate region, and the abnormal part candidate region.

The example in which the detection of the candidate regions from the far-infrared image is realized by performing the process of extracting the partial regions, the process of calculating the score values, and the process of comparing the score values to the thresholds has been described above, but a specific method of detecting the candidate regions is not limited to this example, in a way similar to the first application example and the second application example.

### (Operation)

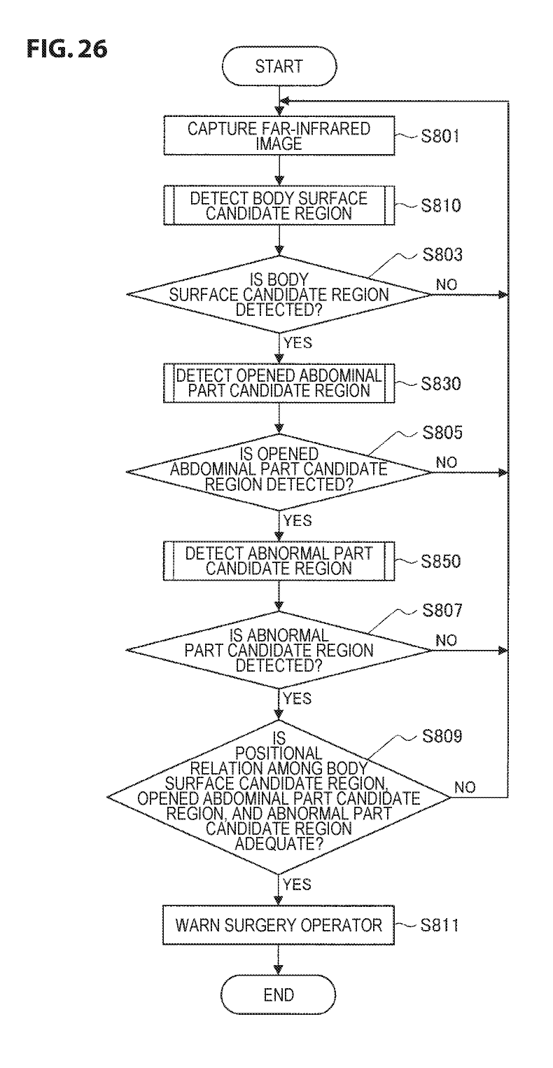

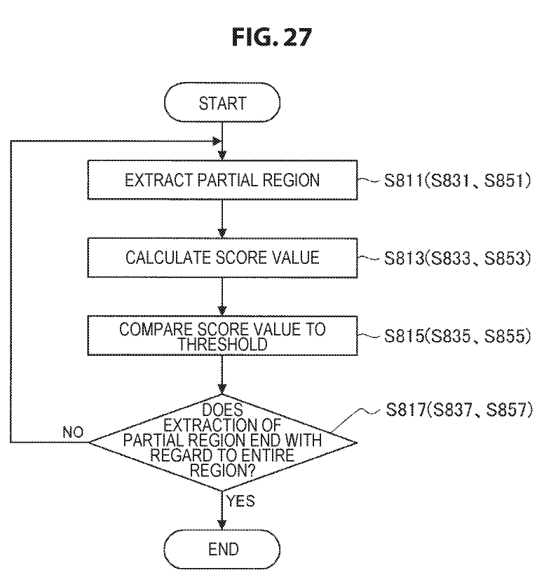

Next, the flow of the process performed by the image processing device 30 according to the third application example will be described with reference to FIGS. 26 and 27. FIG. 26 is a flowchart illustrating an example of the flow of the process performed by the image processing device 30 according to the third application example. The process illustrated in FIG. 26 can be performed on, for example, each frame.

As illustrated in FIG. 26, the image processing device 30 first captures the far-infrared image Im30 (step S801). Subsequently, the body surface detection unit 341 performs a detection process of detecting the body surface candidate region from the captured far-infrared image Im30 (step S810) and outputs a detection result to the determination unit 350. Then, the determination unit 350 determines whether or not the body surface candidate region is detected (step S803). In a case in which it is determined that the body surface candidate region is not detected (NO in step S803), the process returns to step S801.

Conversely, in a case in which it is determined that the body surface candidate region is detected (YES in step S803), a determination result is output from the determination unit 350 to the opened abdominal part detection unit 342. The opened abdominal part detection unit 342 performs a detection process of detecting the opened abdominal part candidate region (step S830) and outputs a detection result to the determination unit 350. Then, the determination unit 350 determines whether or not the opened abdominal part candidate region is detected (step S805). In a case in which it is determined that the opened abdominal part candidate region is not detected (NO in step S805), the process returns to step S801.

Conversely, in a case in which it is determined that the opened abdominal part candidate region is detected (YES in step S805), a determination result is output from the determination unit 350 to the abnormal part detection unit 343. The abnormal part detection unit 343 performs a detection process of detecting the abnormal part candidate region (step S850) and outputs a detection result to the determination unit 350. Then, the determination unit 350 determines whether or not the abnormal part candidate region is detected (step S807). In a case in which it is determined that the abnormal part candidate region is not detected (NO in step S807), the process returns to step S801.

Conversely, in a case in which it is determined that the abnormal part candidate region is detected (YES in step S807), the determination unit 350 determines whether or not the positional relation among the body surface candidate region, the opened abdominal part candidate region, and the abnormal part candidate region is adequate (step S809). In a case in which it is determined that the positional relation among the body surface candidate region, the opened abdominal part candidate region, and the abnormal part candidate region is not substantially identical to the positional relation regulated by the modeling, it is determined that the positional relation among the body surface candidate region, the opened abdominal part candidate region, and the abnormal part candidate region is not adequate (NO in step S809) and the process returns to step S801. Conversely, in a case in which it is determined that the positional relation among the body surface candidate region, the opened abdominal part candidate region, and the abnormal part candidate region is substantially identical to the positional relation regulated by the modeling, it is determined that the positional relation among the body surface candidate region, the opened abdominal part candidate region, and the abnormal part candidate region is adequate (YES in step S809). The determination unit 350 outputs a determination result to the display 108 to warn the surgery operator (step S811) and ends the process illustrated in FIG. 26.

Note that an order of the detection process (step S810) for the body surface candidate region, the detection process (step S830) for the opened abdominal part candidate region, and the detection process (step S850) for the abnormal part candidate region is not limited to this example. In addition, the detection process (step S810) for the body surface candidate region, the detection process (step S830) for the opened abdominal part candidate region, and the detection process (step S850) for the abnormal part candidate region may be performed in parallel.

Next, the detection processes (steps S810, S830, and 850 illustrated in FIG. 26) for the body surface candidate region, the opened abdominal part candidate region, and the abnormal part candidate region performed by the image processing device 30 according to the third application example will be described in more detail with reference to FIG. 27. FIG. 27 is a flowchart illustrating an example of a flow of the detection processes for the body surface candidate region, the opened abdominal part candidate region, and the abnormal part candidate region performed by the image processing device 30 according to the third application example.

As illustrated in FIG. 27, the body surface detection unit 341 (the opened abdominal part detection unit 342, the abnormal part detection unit 343) first extracts a partial region from the far-infrared image Im30 (step S811 (steps S831 and S851)). Subsequently, the body surface detection unit 341 (the opened abdominal part detection unit 342, the abnormal part detection unit 343) calculates a score value of the extracted partial region (step S813 (steps S833 and S853)). Subsequently, the body surface detection unit 341 (the opened abdominal part detection unit 342, the abnormal part detection unit 343) compares the calculated score value to the threshold (step S815 (steps S835 and S855)). Then, the body surface detection unit 341 (the opened abdominal part detection unit 342, the abnormal part detection unit 343) determines whether or not the extraction of the partial region ends with regard to the entire region of the far-infrared image Im30 (step S817 (steps S837 and S857)). In a case in which it is determined that the extraction of the partial region does not end with regard to the entire region of the far-infrared image Im30 (NO in step S817 (NO in step S837 and NO in step S857)), the process returns to step S811 (steps S831 and S851). Conversely, in a case in which it is determined that the extraction of the partial region ends with regard to the entire region of the far-infrared image Im30 (YES in step S817 (YES in step S837 and YES in step S857)), the process illustrated in FIG. 27 ends.

Note that a computer program realizing each function of the image processing device 1 according to the above-described embodiment and the image processing devices 10, 20, and 30 according to the application examples can be produced and mounted on a PC or the like. The image processing device 1 according to the embodiment or the image processing devices 10, 20, and 30 according to the application examples can be equivalent to a computer. In addition, a computer-readable recording medium storing the computer program can also be provided. Examples of the recording medium include a magnetic disk, an optical disc, a magneto-optical disc, a flash memory, and the like. In addition, the foregoing computer program may be delivered via, for example, a network without using a recording medium. In addition, the functions of the image processing device 1 according to the embodiment and the image processing devices 10, 20, and 30 according to the application examples may be divided to a plurality of computers. In this case, functions of the plurality of computers can be realized in accordance with the foregoing computer program.

### 4. CONCLUSION

As described above, according to the embodiment of the present disclosure, whether or not the predetermined subject is shown in the far-infrared image is determined on the basis of the modeling and the positional relation between the plurality of detection regions indicating temperatures within mutually different setting temperature ranges. Therefore, when it is determined whether or not the predetermined subject is shown in the far-infrared image, a more likely determination result can be obtained. Consequently, detection precision of the subject can be improved without using another device different from the infrared camera 102. Accordingly, it is possible to improve the detection precision of the subject at a lower cost.

The examples in which each detection unit extracts the partial region with a predetermined dimension have mainly been described above, but each detection unit may extract a partial region with a plurality of dimensions. Thus, a partial region can be extracted more reliably irrespective of a distance between a target and the infrared camera 102.

Note that the technology according to the above-described present disclosure can be applied to various uses. Specifically, the technology according to the present disclosure can be applied to detect a living body other than a human body. In addition, the image processing device according to the present disclosure can be applied to a vehicle system, a medical system, an automatic production system, and the like.

In addition, the series of control processes by each device described in the present specification may be realized using one of software, hardware, and a combination of the software and the hardware. For example, a program including the software is stored in advance on a storage medium (non-transitory media) provided internally or externally in each device. Then, for example, each program is read to the RAM at the time of execution and is executed by a processor such as the CPU. One processor or a plurality of processors may be provided to execute the respective programs.

Moreover, the process described using the flowchart in the present specification may not necessarily be performed in the order shown in the flowchart. Several processing steps may be performed in parallel. Moreover, additional processing steps may be adopted or some of the processing steps may be omitted.

The preferred embodiment(s) of the present disclosure has/have been described above with reference to the accompanying drawings, whilst the present disclosure is not limited to the above examples. A person skilled in the art may find various alterations and modifications within the scope of the appended claims, and it should be understood that they will naturally come under the technical scope of the present disclosure.

Further, the effects described in this specification are merely illustrative or exemplified effects, and are not limitative. That is, with or in the place of the above effects, the technology according to the present disclosure may achieve other effects that are clear to those skilled in the art from the description of this specification.

Additionally, the present technology may also be configured as below.

(1)

An image processing device including:

a plurality of detection units configured to detect respective detection regions indicating temperatures within mutually different setting temperature ranges from a far-infrared image; and

a determination unit configured to determine whether a predetermined subject is shown in the far-infrared image on the basis of a positional relation between the plurality of detected detection regions and modeling which regulates the positional relation in a case in which the predetermined subject is shown in the far-infrared image.

(2)

The image processing device according to (1),

in which the setting temperature range is a temperature range in accordance with an assumed temperature of a target corresponding to each of the detection units, and

the detection region is equivalent to a candidate region which is a candidate in which the target is shown.

(3)

The image processing device according to (2), in which the detection unit detects a region that has dimensions set in accordance with the target, as the candidate region.

(4)

The image processing device according to (2) or (3), in which the detection unit detects a region indicating a temperature for which likelihood is greater than a threshold, as the candidate region, the likelihood being the assumed temperature of the target.

(5)

The image processing device according to any one of (2) to (4),

in which the plurality of detection units include a face detection unit that detects a face candidate region which is a candidate in which a face of a human body is shown as the candidate region from the far-infrared image and a trunk detection unit that detects a trunk candidate region which is a candidate in which a trunk of the human body is shown as the candidate region from the far-infrared image, and

the determination unit determines whether the human body is shown as the predetermined subject in the far-infrared image on the basis of the modeling and a positional relation between the detected face candidate region and the detected trunk candidate region.

(6)

The image processing device according to (5),

in which the plurality of detection units further include a face part detection unit that detects a face part candidate region which is a candidate in which a part related to the face is shown as the candidate region from the far-infrared image, and

the determination unit determines whether the human body is shown as the predetermined subject in the far-infrared image on the basis of the modeling and a positional relation between the detected face candidate region and the detected face part candidate region.

(7)

The image processing device according to any one of (2) to (4),

in which the plurality of detection units include a muffler detection unit that detects a muffler candidate region which is a candidate in which a muffler of a vehicle is shown as the candidate region from the far-infrared image and a passage detection unit that detects a passage candidate region which is a candidate in which a portion through which a wheel of the vehicle passes on a road surface is shown as the candidate region from the far-infrared image, and

the determination unit determines whether the vehicle is shown as the predetermined subject in the far-infrared image on the basis of the modeling and a positional relation between the detected muffler candidate region and the detected passage candidate region.

(8)

The image processing device according to any one of (2) to (4),

in which the plurality of detection units include a body surface detection unit that detects a body surface candidate region which is a candidate in which a body surface of a patient is shown as the candidate region from the far-infrared image, an incised part detection unit that detects an incised part candidate region which is a candidate in which an incised part of the patient is shown as the candidate region from the far-infrared image, and an abnormal part detection unit that detects an abnormal part candidate region which is a candidate in which an abnormal part is shown in the incised part as the candidate region from the far-infrared image, and

the determination unit determines whether the abnormal part is shown as the predetermined subject in the far-infrared image on the basis of the modeling and a positional relation among the detected body surface candidate region, the detected incised part candidate region, and the detected abnormal part candidate region.

(9)

The image processing device according to any one of (1) to (8), including:

an imaging unit configured to capture the far-infrared image.

(10)

An image processing method including:

detecting respective detection regions indicating temperatures within mutually different setting temperature ranges from a far-infrared image; and

determining whether a predetermined subject is shown in the far-infrared image by an image processing device on the basis of a positional relation between the plurality of detected detection regions and modeling which regulates the positional relation in a case in which the predetermined subject is shown in the far-infrared image.

### REFERENCE SIGNS LIST

1, 10, 20, 30 image processing device
31 microscopic device
31a camera
33 patient bed
35 nonwoven fabric
37 patient
41 first detection unit
41a first extraction unit
41b first score calculation unit
41c first score comparison unit
42 second detection unit
42a second extraction unit
42b second score calculation unit
42c second score comparison unit
50, 150, 250, 350 determination unit
60, 160, 260, 360 storage unit
102 infrared camera
104 input interface
106 memory
108 display
110 communication interface
112 storage
114 processor
116 bus
141 face detection unit
142 trunk detection unit
143 eye detection unit
144 glasses detection unit
145 hair detection unit
241 muffler detection unit
242 passage detection unit
243 non-passage detection unit
341 body surface detection unit
342 opened abdominal part detection unit
343 abnormal part detection unit

1. An image processing device comprising:
a plurality of detection units configured to detect respective detection regions indicating temperatures within mutually different setting temperature ranges from a far-infrared image; and
a determination unit configured to determine whether a predetermined subject is shown in the far-infrared image on a basis of a positional relation between the plurality of detected detection regions and modeling which regulates the positional relation in a case in which the predetermined subject is shown in the far-infrared image.

2. The image processing device according to claim 1,
wherein the setting temperature range is a temperature range in accordance with an assumed temperature of a target corresponding to each of the detection units, and
the detection region is equivalent to a candidate region which is a candidate in which the target is shown.

3. The image processing device according to claim 2, wherein the detection unit detects a region that has dimensions set in accordance with the target, as the candidate region.

4. The image processing device according to claim 2, wherein the detection unit detects a region indicating a temperature for which likelihood is greater than a threshold, as the candidate region, the likelihood being the assumed temperature of the target.

5. The image processing device according to claim 2,
wherein the plurality of detection units include a face detection unit that detects a face candidate region which is a candidate in which a face of a human body is shown as the candidate region from the far-infrared image and a trunk detection unit that detects a trunk candidate region which is a candidate in which a trunk of the human body is shown as the candidate region from the far-infrared image, and
the determination unit determines whether the human body is shown as the predetermined subject in the far-infrared image on a basis of the modeling and a positional relation between the detected face candidate region and the detected trunk candidate region.

6. The image processing device according to claim 5,
wherein the plurality of detection units further include a face part detection unit that detects a face part candidate region which is a candidate in which a part related to the face is shown as the candidate region from the far-infrared image, and
the determination unit determines whether the human body is shown as the predetermined subject in the far-infrared image on a basis of the modeling and a positional relation between the detected face candidate region and the detected face part candidate region.

7. The image processing device according to claim 2,
wherein the plurality of detection units include a muffler detection unit that detects a muffler candidate region which is a candidate in which a muffler of a vehicle is shown as the candidate region from the far-infrared image and a passage detection unit that detects a passage candidate region which is a candidate in which a portion through which a wheel of the vehicle passes on a road surface is shown as the candidate region from the far-infrared image, and
the determination unit determines whether the vehicle is shown as the predetermined subject in the far-infrared image on a basis of the modeling and a positional relation between the detected muffler candidate region and the detected passage candidate region.

8. The image processing device according to claim 2,
wherein the plurality of detection units include a body surface detection unit that detects a body surface candidate region which is a candidate in which a body surface of a patient is shown as the candidate region from the far-infrared image, an incised part detection unit that detects an incised part candidate region which is a candidate in which an incised part of the patient is shown as the candidate region from the far-infrared image, and an abnormal part detection unit that detects an abnormal part candidate region which is a candidate in which an abnormal part is shown in the incised part as the candidate region from the far-infrared image, and
the determination unit determines whether the abnormal part is shown as the predetermined subject in the far-infrared image on a basis of the modeling and a positional relation among the detected body surface candidate region, the detected incised part candidate region, and the detected abnormal part candidate region.

9. The image processing device according to claim 1, comprising:
an imaging unit configured to capture the far-infrared image.

10. An image processing method comprising:
detecting respective detection regions indicating temperatures within mutually different setting temperature ranges from a far-infrared image; and
determining whether a predetermined subject is shown in the far-infrared image by an image processing device on a basis of a positional relation between the plurality of detected detection regions and modeling which regulates the positional relation in a case in which the predetermined subject is shown in the far-infrared image.
